(19) United States
(12) Patent Application Publication
(10) Pub. No.: US 2011/0160613 A1
(43) Pub. Date: Jun. 30, 2011

(54) FLUID COLLECTION/INJECTION DEVICE HAVING SYRINGE SAMPLE CONTAINER AND METHOD OF MAKING AND USING THE SAME

(75) Inventor: Steven SCHRAGA, Surfside, FL (US)

(73) Assignee: STAT MEDICAL DEVICES, INC., North Miami, FL (US)

(21) Appl. No.: 12/974,908

(22) Filed: Dec. 21, 2010

Related U.S. Application Data
(60) Provisional application No. 61/289,178, filed on Dec. 22, 2009.

(51) Int. Cl. A61B 5/15

(52) U.S. Cl. 600/576

(57) ABSTRACT

Fluid collection/injection device/system includes a fluid sampling syringe having a double-ended needle arranged on a forward end and a rear end. A syringe sampling device has a forward end insertable into the rear end of the fluid sampling syringe. The forward end of the syringe sampling device being puncturable by one needle of the double-ended needle.

### CROSS-REFERENCE TO RELATED APPLICATIONS

The instant application is a US non-provisional Application based on U.S. provisional application No. 61/289,178, filed Dec. 22, 2009, the disclosure of which is hereby expressly incorporated by reference hereto in its entirety.

### BACKGROUND OF THE INVENTION

1. Field of the Invention

This invention relates generally to devices used to collect fluid samples from and/or inject fluids into patients. More specifically, this invention relates to a device which utilizes a holder having a double-ended needle that can be released, removed from and/or retracted into the holder in a more safe and easy manner. The invention also relates to a device which utilizes a holder having a double-ended needle and that utilizes a syringe sample container instead of an evacuated collection tube which is typically utilized in Vacutainer type devices. The device can be a single-use device. The invention also relates to a method of collecting a fluid sample with the device as well as a method of making the device. The invention also relates to a blood sample collection device that is less costly to produce and/or easier to manufacture. Finally, the invention relates to a device that can be used to inject one or more substances.

2. Discussion of Background Information

Prevention of needle sticks is of paramount concern in the healthcare industry because of serious and deadly risk factors associated with AIDS and other serious communicable diseases. Typical blood collection devices utilize a needle inserted into a patient's vein so as to draw blood through the needle into an associated separate collection reservoir. Accidental needle sticks from previously used needles can occur during the fluid withdrawing process and subsequent handling and disposal operation. Until such used medical devices are destroyed, they remain a risk to those handling them.

Devices used for blood sampling are well known and include a collection device sold under the trademark Vacutainer® by Becton Dickinson Corporation. This device has a tubular syringe-like body with a needle in the front end, part of which extends back into a tubular syringe-like shell. Part of the needle extends externally for punching the skin. An evacuated collection tube with a rubber stopper is placed into the open back of the syringe-like shell with the rubber stopper against the internal end of the needle. After the skin is punctured, the collection tube is pushed forward to cause the needle to enter the evacuated tube. Vacuum helps draw blood into the collecting tube. When a sufficient sample has been obtained, the collecting tube and the stopper are simply withdrawn from the tubular shell and sent to the laboratory. This particular device has a permanently extended needle and an opening in the back for the collection tube which remains open after the collection tube is removed, leaving small quantities of blood and an internally exposed needle.

Medical devices which are used for collecting fluid samples from patients which have quick release needle systems are also known. Such devices include: U.S. Pat. No. 5,797,490 to FUJI et al; U.S. Pat. No. 5,755,673 to KINSEY; U.S. Pat. No. 4,822,343 to BEISER; U.S. Pat. No. 4,984,580 WANAMAKER; U.S. Re. 38,964 to SHILLINGTON; U.S. Pat. No. 5,616,136 to SHILLINGTON et al.; U.S. Pat. No. 5,637,101 to SHILLINGTON; U.S. Pat. No. 5,117,837 to WANAMAKER et al.; U.S. Pat. No. 4,907,600 to SPENCER; U.S. Pat. No. 4,993,426 to SPENCER; U.S. Pat. No. 4,904,244 to HARSH et al.; U.S. Pat. No. 4,490,142 to SILVERN. The disclosures of each of these documents is expressly incorporated by reference herein in their entireties.

The invention aims to improve devices of the type described above by making a fluid collection holder which is easier to make and use and/or cheaper to make so that its use can be more widespread. The device is also believed to be as safe or safer to use and/or dispose-of than the above-noted devices. The invention also relates to a fluid sampling device of the type known in the art, but which utilizes a syringe sample container instead of an evacuated collection tube which is typically utilized in Vacutainer type devices. The invention also aims to use a fluid sampling device of the type disclosed in U.S. Provisional Application 61/167,718 to SCHRAGA and/or US 2008/0262421 to SCHRAGA, but which utilizes a syringe sample container instead of an evacuated collection tube which is typically utilized in Vacutainer type devices. The disclosures of each of these two documents is expressly incorporated by reference herein in their entireties. Finally, the invention relates to a device that can be used to inject one or more substances.

### SUMMARY OF THE INVENTION

According to one non-limiting aspect of the invention there is provided a fluid collection/injection device comprising a body having a front end, a back end, and a main hollow section arranged between the front and back ends, a needle hub securing section arranged on the front end and being structured and arranged to receive therein a needle member, a double-ended needle removably and/or movably coupled to the needle hub securing section, and a syringe sampling device having an insertable end which is puncturable by one needle of the double-ended needle.

The syringe sampling device may comprise a body and a plunger having a forward end in sealing engagement with an inner surface of the body. The plunger may have a rear end configured to allow a user to move the plunger in a rear-ward direction so as to limit and/or regulate an amount of fluid that is withdrawn with the device. The plunger may have a piston and a breakable and/or frangible section. The insertable end of the syringe sampling device may comprise a re-sealable member that prevents fluid from leaking out of the syringe sampling device when the one needle is withdrawn. The syringe sampling device may comprise a plunger having a piston and a plurality of breakable and/or frangible sections. The syringe sampling device may comprise a mechanism that prevents forward movement of a plunger and allows rearward movement of the plunger. The syringe sampling device may comprise a ratchet mechanism that prevents forward movement of a plunger and allows rearward movement of the plunger. The insertable end of the syringe sampling device may comprise a removable re-sealable member that prevents fluid from leaking out of the syringe sampling device when the one needle is withdrawn.

The body, the needle hub securing section, and the double ended needle may be a conventional device. The body, the needle hub securing section, and the double ended needle may be of the type disclosed in U.S. Provisional Application 61/167,718 to SCHRAGA. The body, the needle hub securing section, and the double ended needle may be of the type disclosed in US 2008/0262421 to SCHRAGA.

The invention also provides for a method of taking a fluid sample using the device described above, wherein the method comprises inserting the syringe sampling device into the body and removing the syringe sampling device from the body.

The invention also provides for a method of taking a fluid sample using the device described above, wherein the method comprises inserting the syringe sampling device into the body, withdrawing a plunger of the syringe sampling device by a desired amount, and removing the syringe sampling device from the body.

The invention also provides for a fluid collection/injection device comprising a fluid sampling syringe having a double-ended needle arranged on a forward end and a rear end and a syringe sampling device having a forward end insertable into the rear end. The forward end of the syringe sampling device is puncturable by one needle of the double-ended needle.

The fluid sampling syringe can be a conventional device. The fluid sampling syringe may be of the type disclosed in U.S. Provisional Application 61/167,718 to SCHRAGA. The fluid sampling syringe may be of the type disclosed in US 2008/0262421 to SCHRAGA.

The invention also provides for a method of taking a fluid sample using the device described above, wherein the method comprises inserting the syringe sampling device and removing the syringe sampling device.

The invention also provides for a method of taking a fluid sample using the device described above, wherein the method comprises inserting the syringe sampling device, withdrawing a plunger of the syringe sampling device by a desired amount, and removing the syringe sampling device. The invention also provides for a method of making a fluid sample devise shown and/or described herein.

Other exemplary embodiments and advantages of the present invention may be ascertained by reviewing the present disclosure and the accompanying drawing.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 shows a side cross-section view of a first non-limiting embodiment of a fluid sampling device used in the fluid sampling system according to the invention. The needle member is shown in an installed condition and is not shown in cross-section;

FIGS. 2 and 3 show side views of a needle member that can be used in the fluid sampling device of FIG. 1. In FIG. 2, the needle member is shown in an original position with its rear needle covered. In FIG. 3, the needle member is shown in a use position with its rear needle cover in a retracted position exposing the tip of the rear needle. This occurs when the rear needle is caused to puncture a sampling container/device of the type described below after it is inserted into the sampling device shown in FIG. 1;

FIG. 4 shows a side view of the fluid sampling device of FIG. 1, but rotated about 90 degrees and with the needle member removed. FIG. 4 also shows the releasable securing mechanism in a pre-bent configuration;

FIG. 5 shows a side view of the fluid sampling device of FIG. 4, but rotated about 90 degrees, and showing the releasable securing mechanism deflected outwardly by a small amount;

FIG. 6 shows a side view of the fluid sampling device of FIG. 1, but with the needle member removed. In FIG. 6, the free end of the releasable securing mechanism has been bent about 90 degrees from the position shown in FIG. 5 to that shown in FIG. 6;

FIG. 7 shows a front end view of the fluid sampling device of FIG. 6, except that the rear flange of the fluid sampling device is not illustrated. As was the case in FIG. 6, the releasable securing mechanism is biased to the closed position;

FIG. 8 shows a cross-section view of the fluid sampling device of FIG. 8;

FIG. 9 shows a front end view of the fluid sampling device of FIG. 8. The rear flange of the fluid sampling device is not illustrated;

FIG. 10 shows a side view of the fluid sampling device of FIG. 6, but with the releasable securing mechanism being disposed in the open position. The open position occurs when a force is applied to a portion of the releasable securing mechanism sufficient to deflect it from an original position. In this open position, the needle member can be removed and/or installed onto/into the fluid sampling device;

FIG. 11 shows a front end view of the fluid sampling device of FIG. 10. The rear flange of the fluid sampling device is not illustrated;

FIG. 12 shows a cross-section view of the fluid sampling device of FIG. 10;

FIG. 13 shows a front end view of the fluid sampling device of FIG. 12. The rear flange of the fluid sampling device is not illustrated;

FIG. 14 shows a side view of the fluid sampling device of FIG. 4, except that a free end of the releasable securing mechanism is removed to better illustrate a front end of the fluid sampling device;

FIG. 15 shows an enlarged portion of a front end of FIG. 8, except that a free end of the releasable securing mechanism is removed to better illustrate a front end of the fluid sampling device;

FIG. 16 shows a front end view of the fluid sampling device of FIG. 15;

FIG. 17 shows an enlarged portion of FIG. 8, except that front and rear ends of the body of the fluid sampling device are removed to better illustrate the releasable securing mechanism;

FIG. 18 shows a side view of a fluid sample container in accordance with one non-limiting embodiment of the invention. The body and puncturable front cover are shown in cross-section. The plunger is not shown in cross-section and is shown in an initial and/or fully depresses and/or pre-withdrawn position;

FIG. 19 shows how the fluid sample container of FIG. 18 can be inserted into the device of FIG. 1. In FIG. 19, the container of FIG. 18 is only partially inserted into the device of FIG. 1 and has not yet reached the fully inserted position;

FIG. 20 shows the fluid sample container of FIG. 18 in an initial position;

FIG. 21 shows the fluid sample container of FIG. 20 with the plunger in a nearly fully withdrawn position. This would typically occur after the container shown in FIG. 20 is fully installed in the device of FIG. 1. When the user withdraws the plunger, fluid is caused to flow into the container;

FIG. 22 shows a side cross-section view of the body of the fluid sample container of FIG. 20;

FIG. 23 shows a side cross-section view of the puncturable front cover or cap of the fluid sample container of FIG. 20;

FIG. 24 shows a side view of the plunger of the fluid sample container of FIG. 20;

FIG. 25 shows an enlarged partial view of a front portion of another embodiment of the fluid sample container in accordance with the invention. This embodiment utilizes an arrangement for more securely retaining the puncturable front cover on the body. The arrangement includes a projection and recess connection and a retaining ring;

FIG. 26 shows a partial side-cross section view another non-limiting embodiment of a fluid sampling system in accordance with the invention. The needle member and plunger are not shown in cross-section. In FIG. 26, the fluid sampling container is fully inserted into the device and the rear needle of the needle member has punctured the front cover. In this configuration, the user can withdraw the plunger and cause fluid to pass or flow into the container in the same way as a syringe. This embodiment utilizes a stop shoulder to limit insertion movement of the container. The container body also includes a stop surface to limit forward movement of the plunger and provide sufficient spacing so that the rear needle of the needle member does not contact the piston of the plunger;

FIG. 27 shows a cross-section view of still another embodiment of the blood collection device in accordance with the invention. The device utilizes an annular outwardly tapered projection on an inside surface of the outer body which engages with an inwardly tapered leading end of the movable member to cause the front or proximal end of the movable member to expand radially when the movable member moves relative to the needle holding member. This would occur when the cap is moved to the closed position;

FIG. 28 shows a side cross-section view of the needle holding member and a conventional type double-ended needle mounted thereto and used in the embodiment shown in FIG. 27;

FIG. 29 shows a side view of another embodiment of a needle holding member with a conventional type double-ended needle mounted thereto and which can be used in the embodiment shown in FIG. 27;

FIG. 30 shows a side view of still another embodiment of a needle holding member with a conventional type double-ended needle mounted thereto and which can be used in the embodiment shown in FIG. 27;

FIG. 31 shows a side cross-section view of the movable member used in the embodiment shown in FIG. 27;

FIG. 32 shows a side view of the movable member shown in FIG. 31;

FIG. 33 shows a side cross-section view of the tubular outer body used in the embodiment shown in FIG. 27;

FIG. 34 shows an enlarged top cross-section view of a portion of the tubular outer body used in the embodiment shown in FIG. 27;

FIG. 35 shows how the fluid sample container of FIG. 18 can be inserted into the device of FIG. 27. In FIG. 35, the container of FIG. 18 is only partially inserted into the device of FIG. 27 and has not yet reached the fully inserted position;

FIG. 36 shows a side view of a fluid sample container in accordance with another non-limiting embodiment of the invention. The body and puncturable front cover are shown in cross-section. The plunger is not shown in cross-section and is shown in an initial and/or fully depresses and/or pre-withdrawn position. In this embodiment, the plunger has a number of substantially equally spaced breakable or frangible sections as well as indicia so as to provide the user with information as to the quantity of fluid arranged within and/or suctioned into the container during withdrawal of the plunger;

FIG. 37 shows a side view of a fluid sample container in accordance with still another non-limiting embodiment of the invention. The body, the puncturable front cover, the plunger flange, and the ratchet pawl member are shown in cross-section. The plunger is not shown in cross-section and is shown in an initial and/or fully depresses and/or pre-withdrawn position. In this embodiment, the plunger has a number of ratchet teeth which are engaged by pawls so as to prevent the plunger from being depressed after being withdrawn. This provides a one-way movement mechanism and/or ensures that a patient is not accidentally injected with fluid that is being the withdrawn;

FIG. 38 shows a side cross-section view of still another embodiment of the blood collection device in accordance with the invention. The device utilizes an annular outwardly tapered projection on an inside surface of the outer body which engages with an inwardly tapered leading end of the movable member to cause the front or proximal end of the movable member to expand radially when the movable member moves relative to the needle holding member. This would occur when the cap is moved to the closed position;

FIG. 39 shows an enlarged cross-section view of a portion of FIG. 33 and before the movable member is caused to release from engagement with the needle holding member; and

FIG. 40 shows an enlarged cross-section of a front portion of the inner sleeve that can be used in the embodiment of FIG. 38.

### DETAILED DESCRIPTION OF THE INVENTION

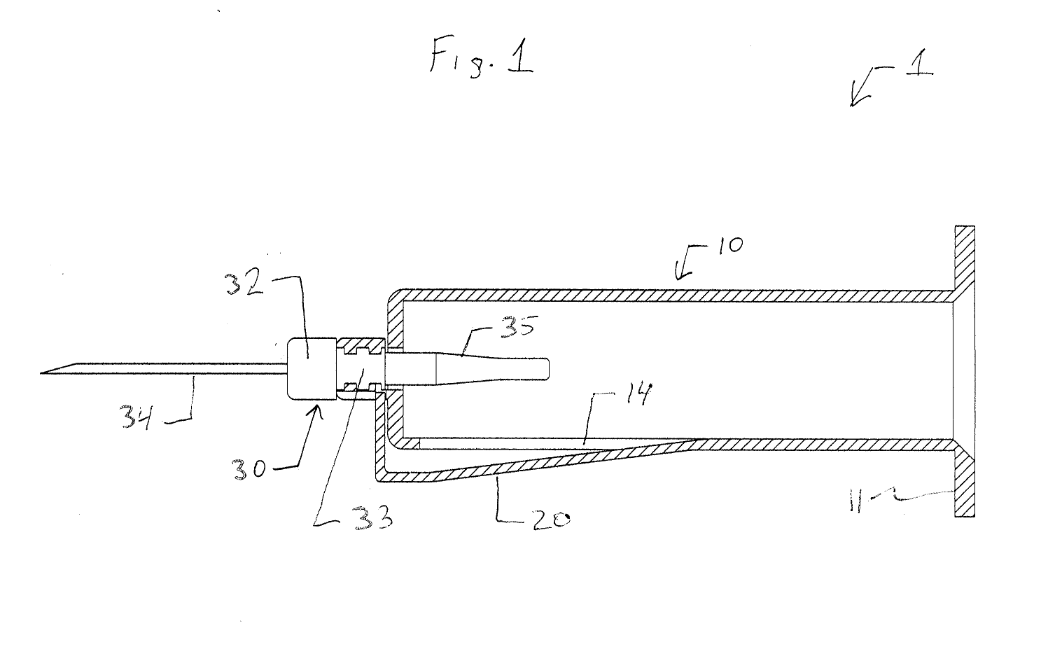

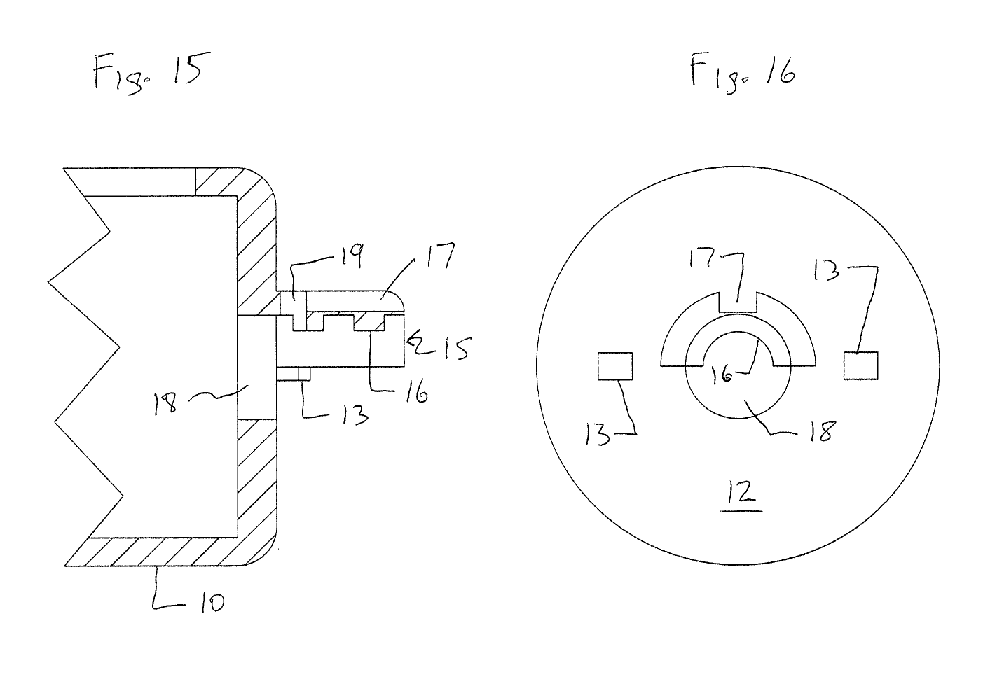

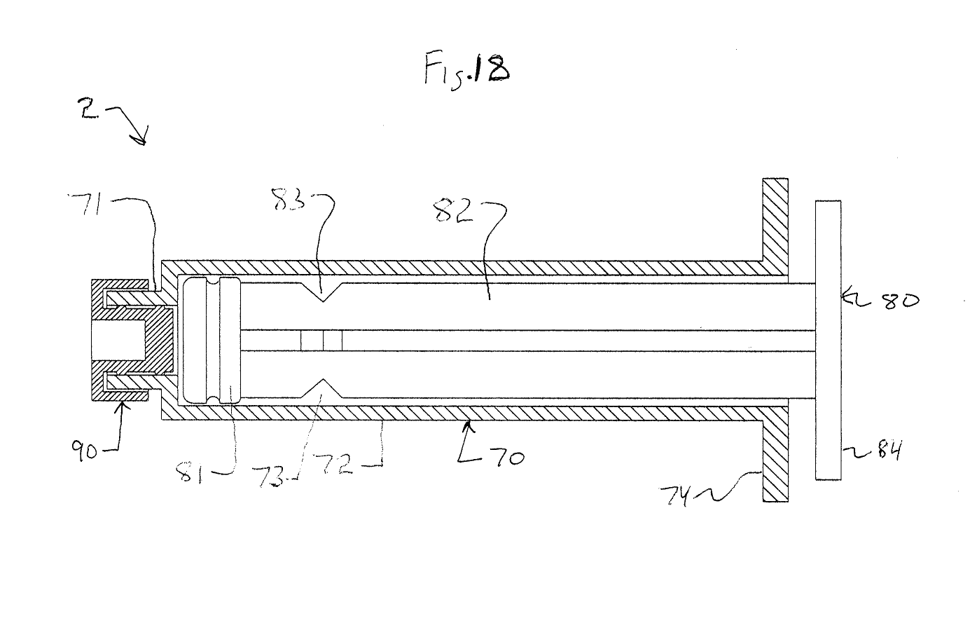

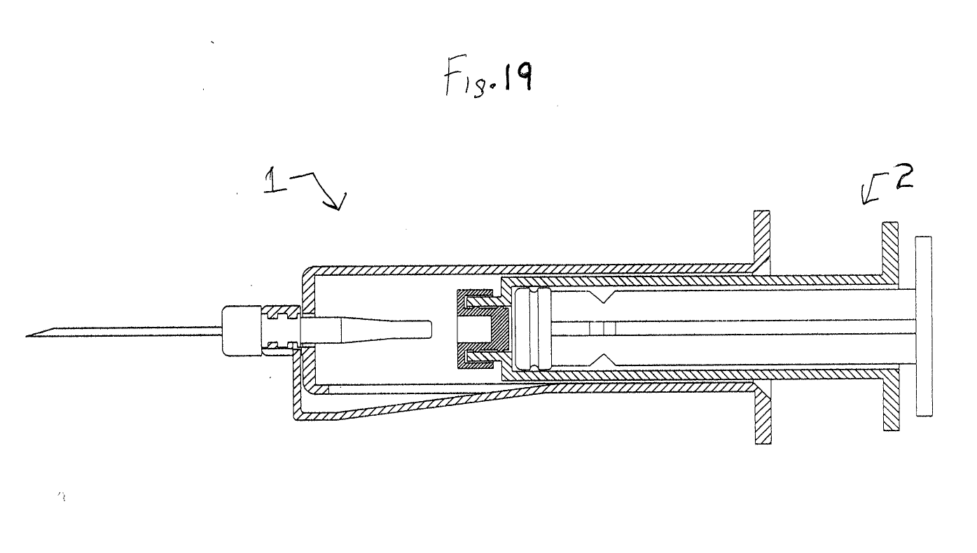

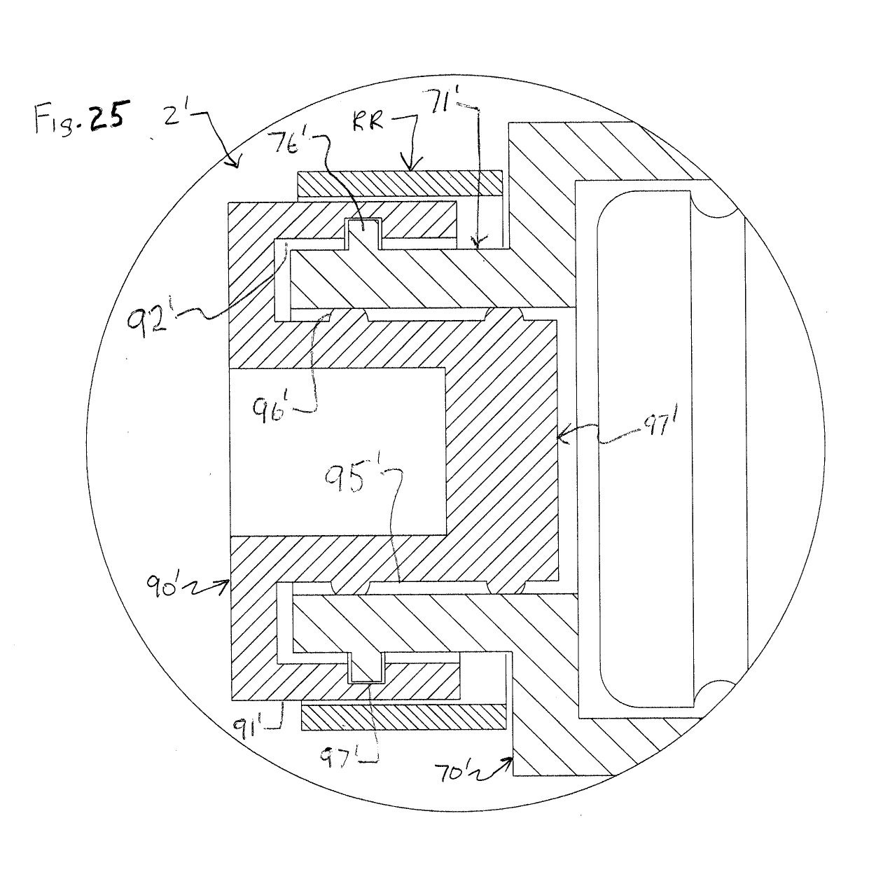

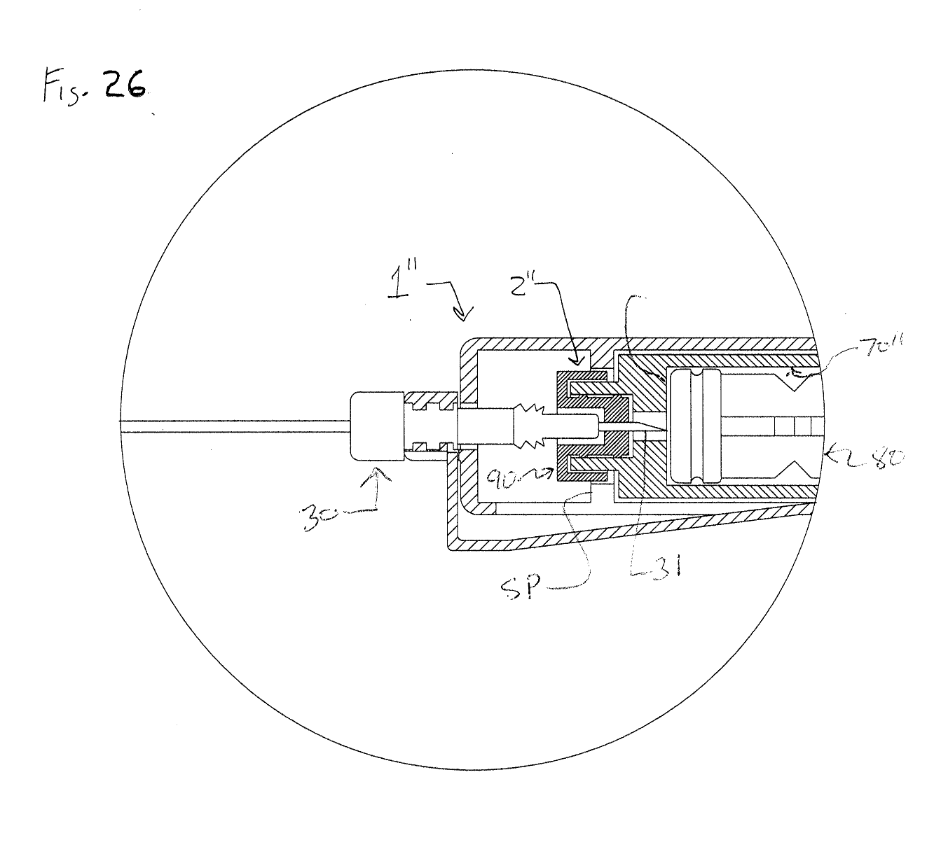

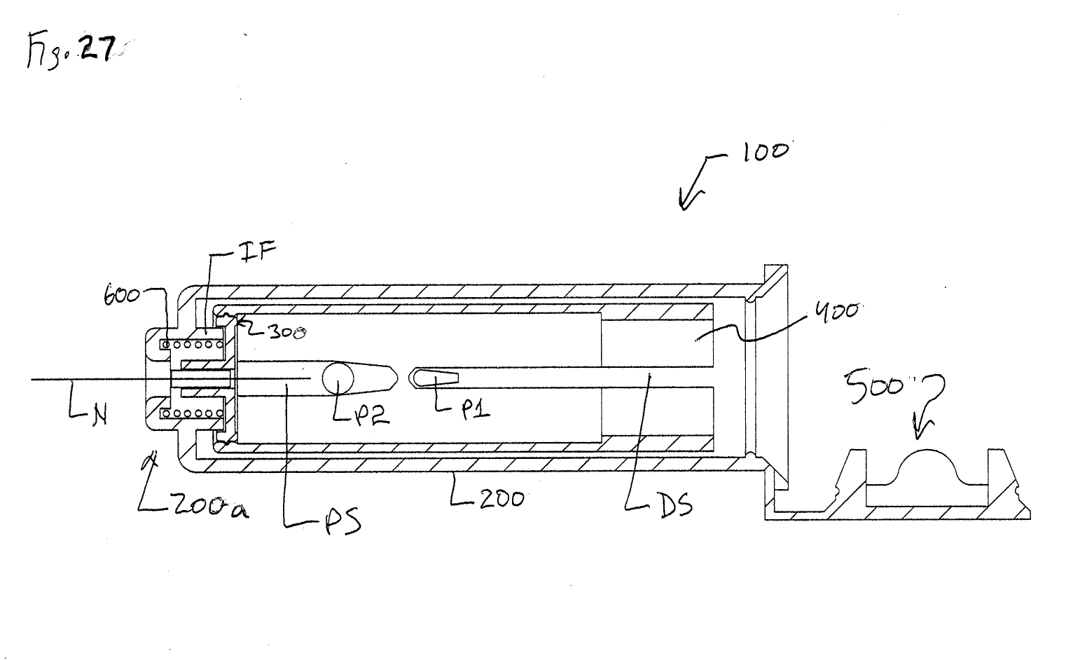

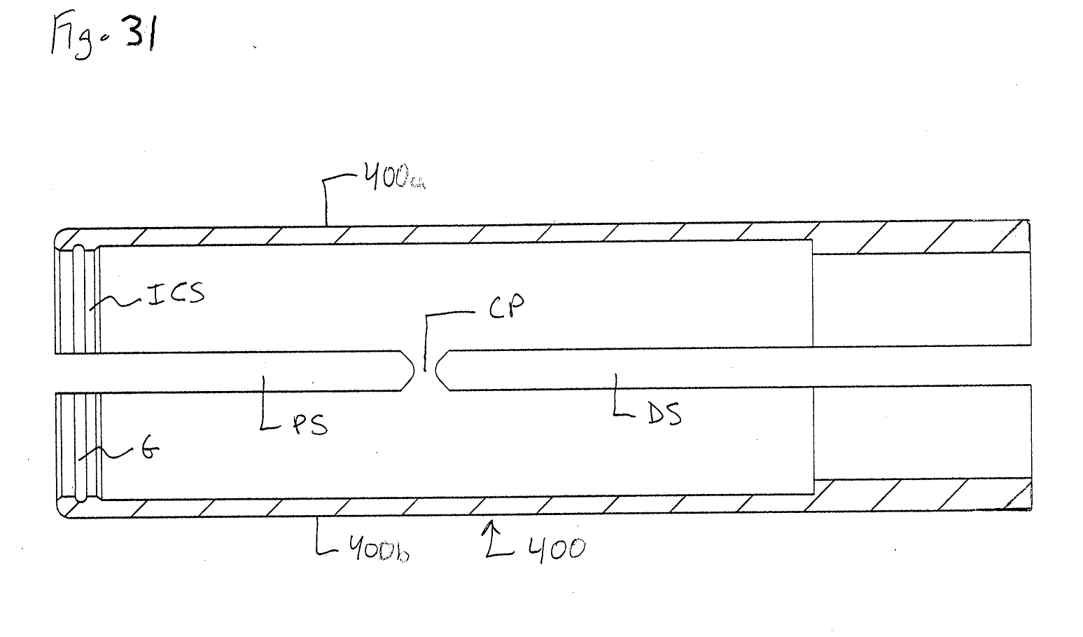

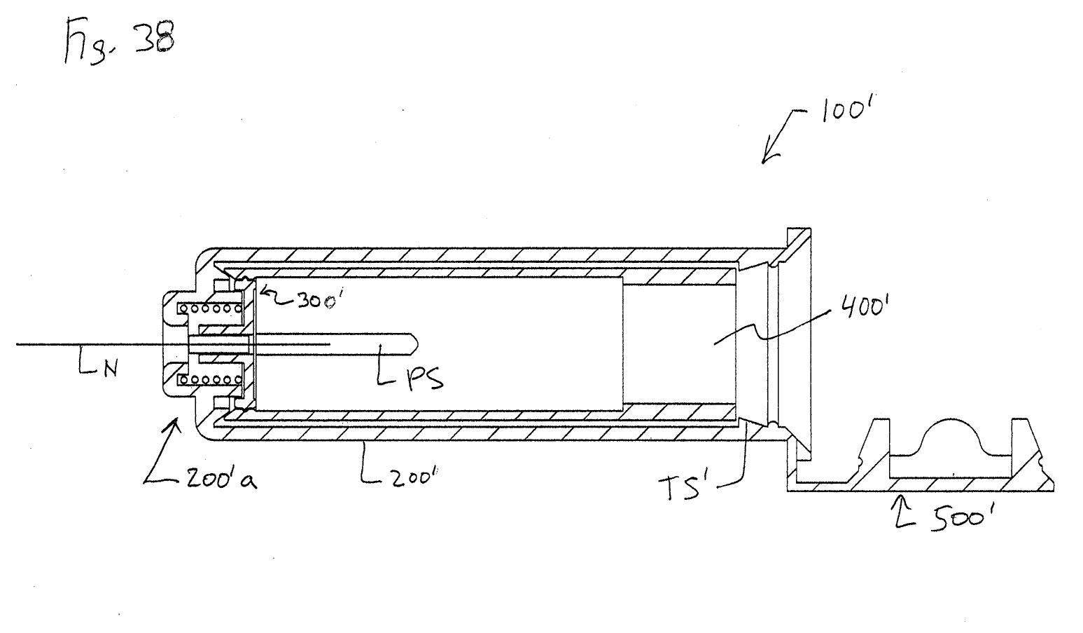

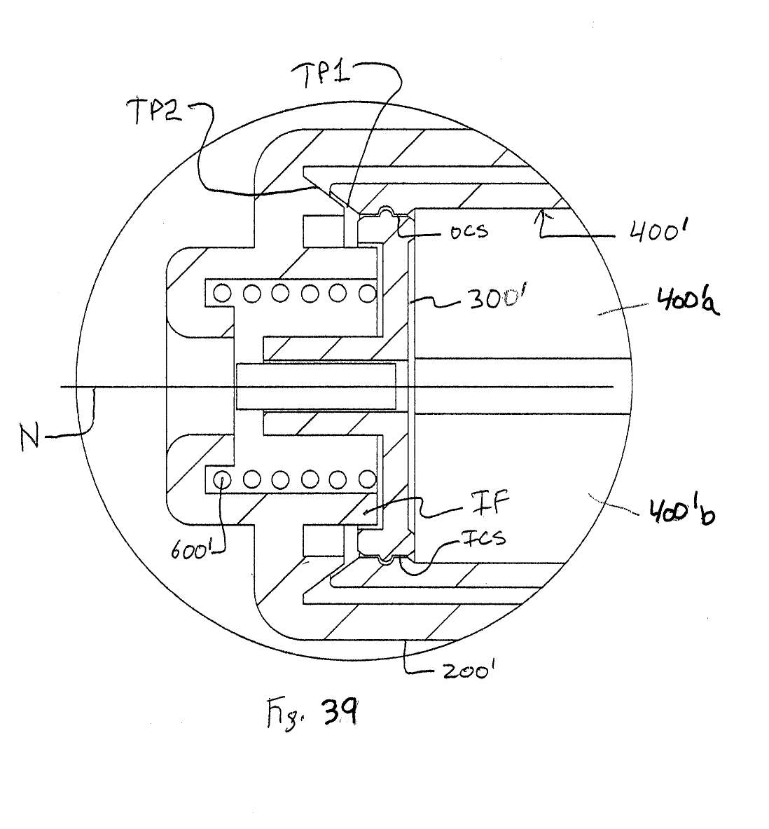

Referring now to the drawings and first to FIGS. 1-23 which shows a first non-limiting embodiment of a system utilizing a device 1 for injection and/or fluid collection and a fluid sampling container 2 according to the invention. The device 1 includes a generally cylindrical sleeve or body member 10 which includes a proximal end configured to allow an external needle 34 of a double-ended needle member or needle holder 30 to pass there through, and a distal end which can receive a collection container such as a vacutainer-type vial. In the embodiment of FIGS. 1-17, the distal end is open. However, it may also be closed off by a cap similar to the device shown in, e.g., FIGS. 27 and 38, and/or disclosed in US 2008/0262421 (U.S. Ser. No. 11/738,240) to SCHRAGA, the entire disclosure of which is hereby expressly incorporated by reference in its entirety. An external flange 11 is arranged at the distal end.

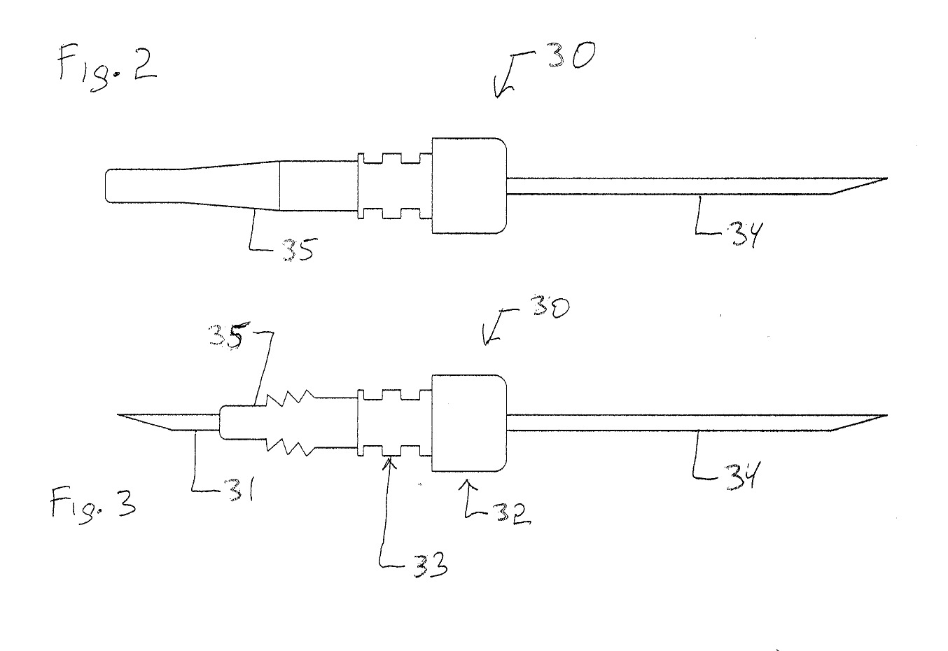

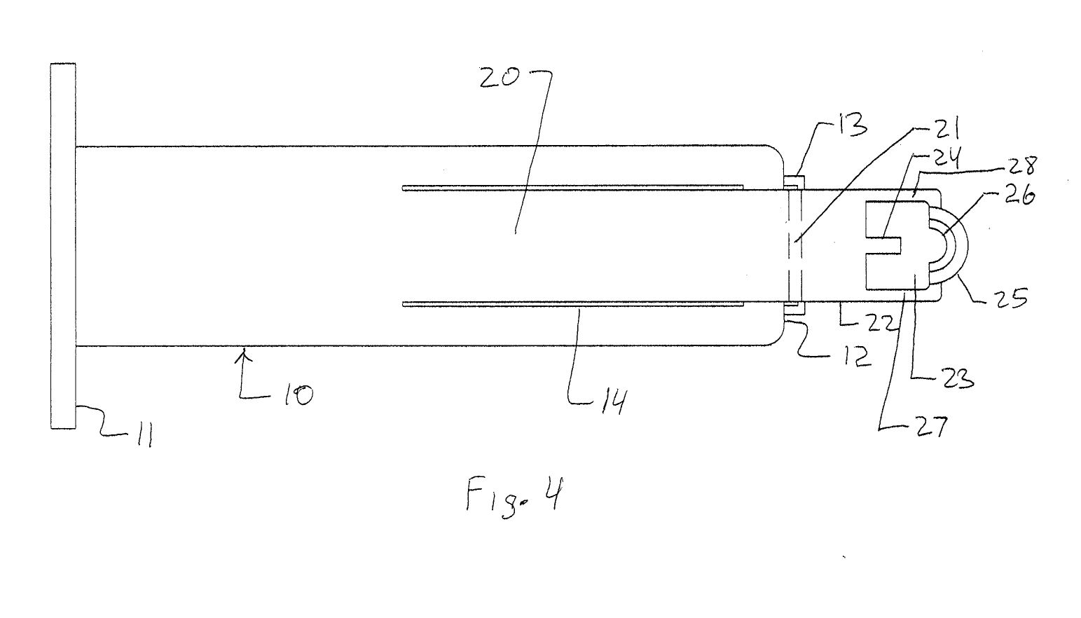

With reference to FIGS. 2 and 3, the needle holder or member 30 can be of any conventional type which can used with the type of device shown in FIG. 1. In embodiments, the member 30 includes a proximal needle 34, a distal needle 31, a retractable/protective cover member 35 and a hub section 32 from which the two needles 31 and 34 extend in opposite directions. The hub section 32 is sized and configured, e.g., has external thread-type feature, which can frictionally engage with inner comparable feature (which is described in detail below) so as to ensure that the member 30 is securely and axially retained when installed on the device (see FIG. 1).

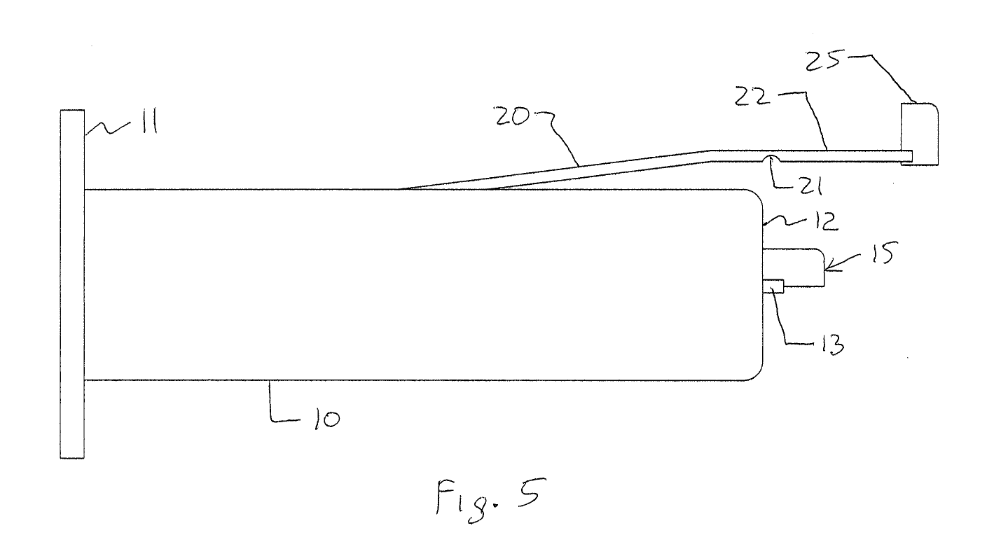

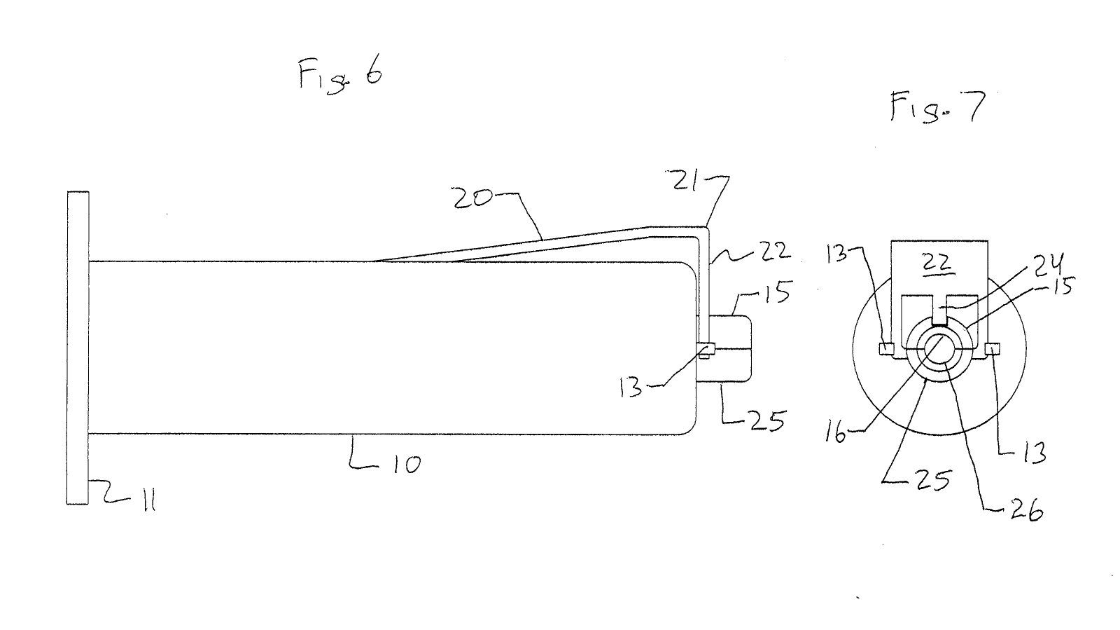

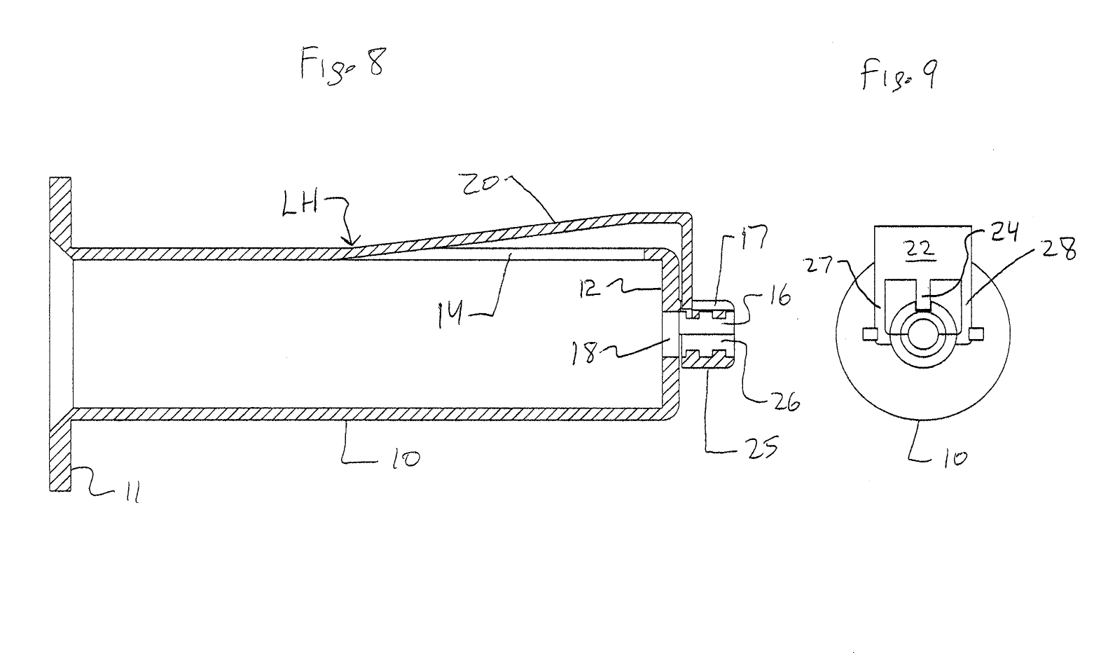

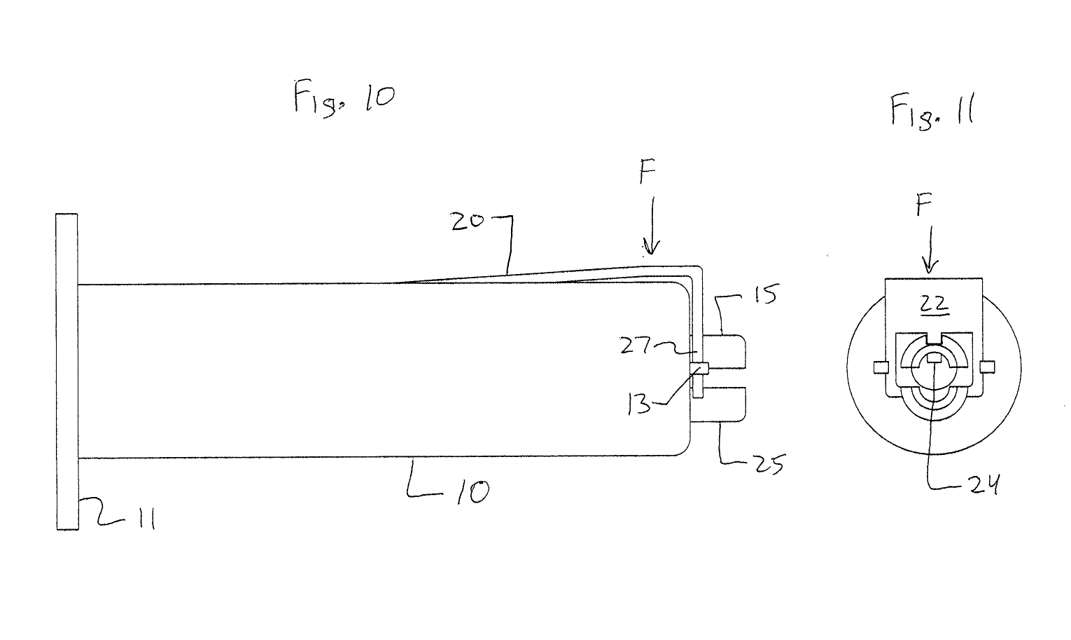

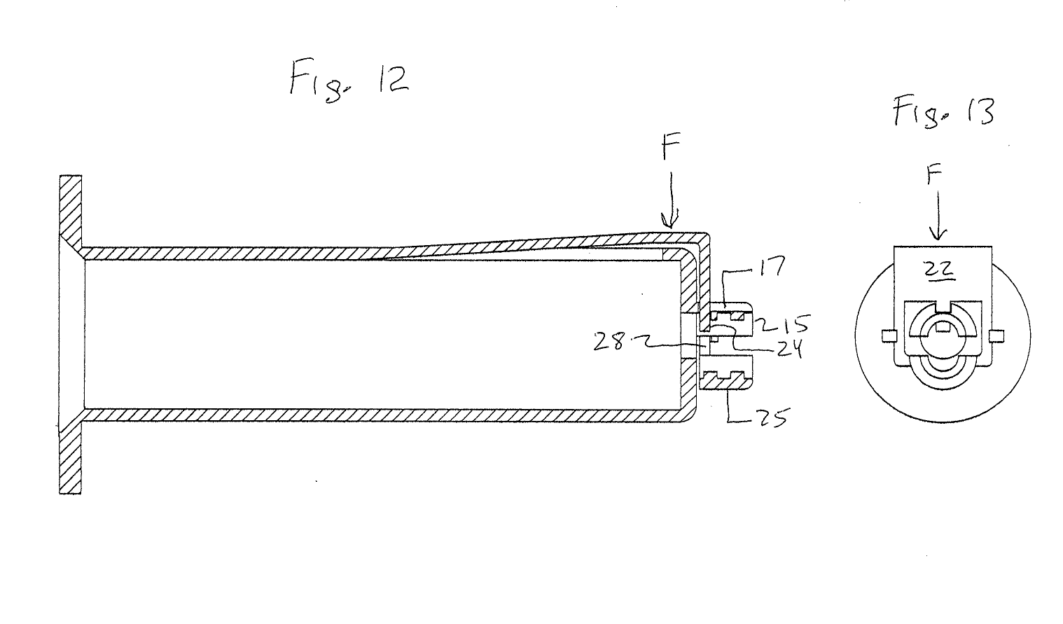

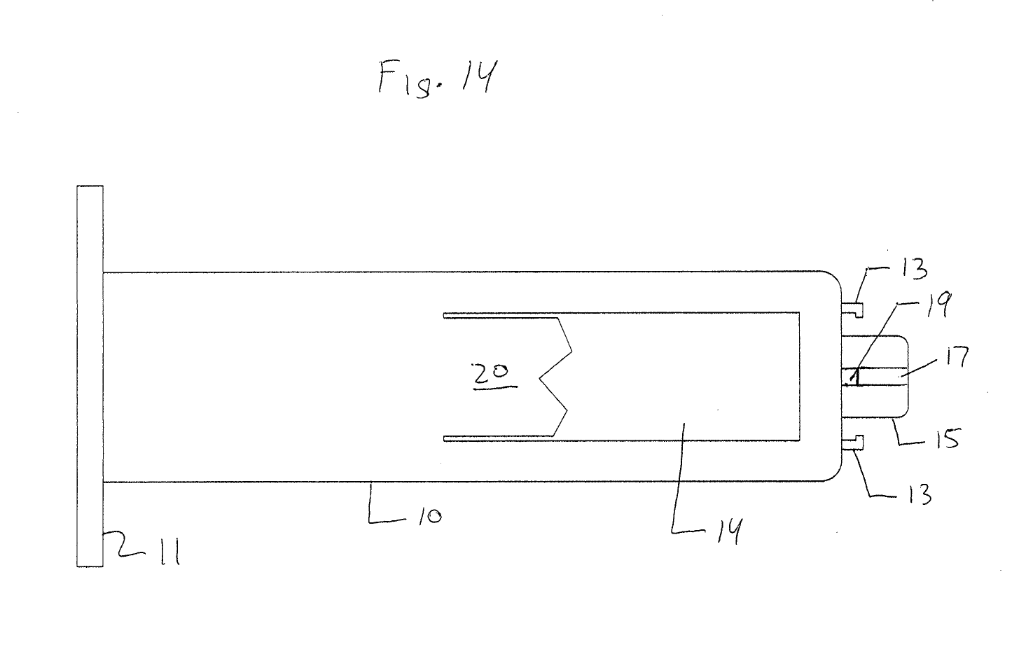

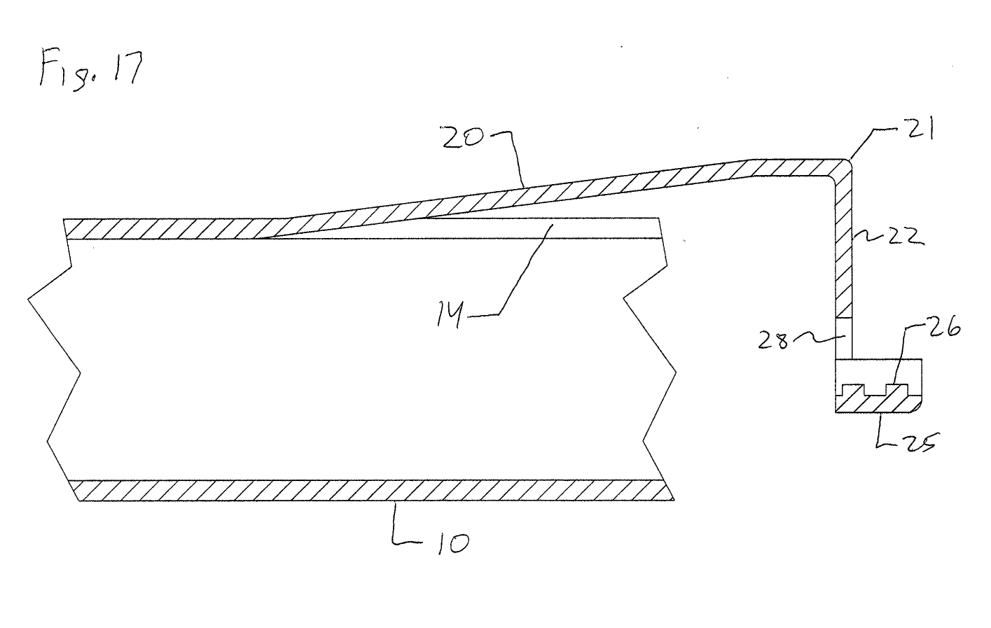

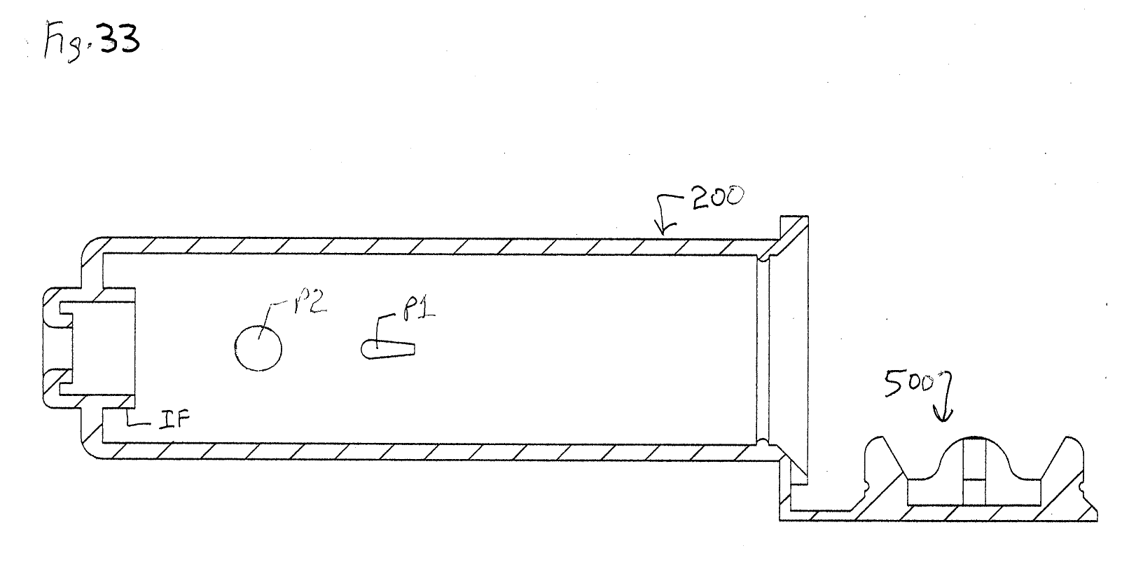

The device 1 has a needle hub securing section arranged on the front or proximal end thereof. This section is structured and arranged to receive therein the needle member 30. The needle hub securing section comprises a fixed part 15 and a movable part 25. In embodiments, the fixed part 15 is integrally formed with the front end of the body 10. The movable part 25 is arranged on a free end of a member 20. In embodiments, the movable part 25 is integrally formed with the member 20. In embodiments, the other end of the member 20 is fixed to a portion of the main hollow section or body 10. In other embodiments, the other end of the member 20 is connected to a portion of the main hollow section 10 via a living hinge section LH (see FIG. 8). In embodiments, the other end of the member 20 is removably connected to a portion of the main hollow section 10 (see e.g., embodiment of FIGS. 30 and 31 of U.S. Ser. No. 12/755,917 to SCHRAGA, the disclosure of which is hereby expressly incorporated by reference in its entirety). In embodiments, the other end of the member 20 is integrally formed with the main hollow section 10 as is utilized in the embodiment of FIGS. 1-17. The deflectable member 20 includes a movable connecting section 22. In embodiments, the section 22 is fixed to a portion of the member 20. In other embodiments, the section 22 is connected to a portion of the member 20 via a living hinge 21. As is apparent from a comparison of FIG. 5 and FIGS. 6 and 7, the section 22 can be bent over approximately 90 degrees and fillet into and between to retaining guides 13 arranged on a front end of the body 10. A projection 24 is arranged on or coupled to the section 22 (see FIG. 7). As is apparent from FIGS. 9 and 11, the projection 24 is configured to extend into an opening 19 (see FIG. 14) formed in the fixed part 15. The purpose of the projection 24 is to cause ejection of the member 40 when the deflectable member 20 is moved from an original position shown in FIGS. 6-9 to the ejection position shown in FIGS. 10-13.

With reference to FIGS. 6-9, it can be seen that the natural elasticity of the member 20 ensures that the section 22 is in an original position. As a result, the movable part 25, which is coupled thereto, is in a closed position by virtue of being in contact with the fixed part 15. These parts 15 and 25 are essentially semi-circular and when placed in contact with one another form an opening comprised of two semi-circular engaging sections 16 and 26. It is these sections 16 and 26 which frictionally engage with the engagement section 43 of the member 40. In order to install the member 40, so as to place the device 1 in the usable configuration of FIG. 1, the user applies a force F (see FIGS. 10-13) to the member 20 which causes the movable part 25 to move away from the closed position to the open position. This movement is a linear movement as a result of the sliding engagement between portions 27 and 28 of the section 22 and retaining guides 13. The retaining guides 13 have an upper or proximal shoulder (see FIG. 14) which ensures that section 22 (and specifically sections 27 and 28) maintain the bent configuration and prevent them from moving back to the position shown in FIGS. 4 and 5. At this point, a user can install the member 40 onto the body 10 by inserting the end 45 into the enlarged space formed between parts 15 and 25 until the annular surface of the section 42 contacts an annular proximal surface of the parts 15 and 25. The user can then remove the force F and allow the movable part 25 to again assume the position shown in FIGS. 6-9, which results in the device assuming the configuration of FIG. 1.

With reference to FIGS. 6-17, it can be seen that the body 10 has a side opening 14 which allows for deflection movement of the member 20 noted above. An annular surface 12 defines a proximal end of the space formed in the section 10. A centrally disposed opening 18 allows the section 45 of the member 40 to pass into the space. As should be apparent from FIGS. 9, 11 and 13, in order to bend the section 22 90 degrees without damaging the projection 24, a slot or recess 17 is formed in the fixed part 15. To retain the section 22 in the bent configuration, the retainer guides 13 trap sections 27 and 28 there between.

The operation of the device of FIGS. 1-17 will now be described. According to one non-limiting embodiment, the device shown in FIGS. 4 and 5 is packaged. Once the package is open, a user bends section 22 along living hinge 21 and slightly deflects sections 27 and 28 until the section 22 is arranged between retaining guides 13 resulting in the configuration shown in FIGS. 6 and 7. Then, the user installs the needle member 40 so that the device 1 assumes the configuration of FIG. 1. This is accomplished when the user applies a force F (see FIGS. 10-13) to the member 20 which causes the movable part 25 to move away from the closed position to the open position. At this point, the user can install the member 40 onto the body 10 by inserting the end 45 into the enlarged space formed between parts 15 and 25 and into opening 18, and until the annular surface of the section 42 contacts an annular proximal surface of the parts 15 and 25. The user can then remove the force F and allow the movable part 25 to again assume the position shown in FIGS. 6-9, which results in the device assuming the configuration of FIG. 1. The user can then remove a safety cover (not shown) from the proximal needle 34, inject the same, e.g., into a human or animal, and then, e.g., insert a vacutainer-type vial into the space formed in the body 10. Once the device 1 is utilized to obtain a sample of desired amount and after the sample vial is removed from the interior space of the body 10, and after the injection needle 34 is removed from the injection site, the user can again apply the force F and move the movable part 25 to the open position shown in FIGS. 10-13. The mere opening of the space between parts 15 and 25 should allow the member 30 to drop out of the device 1. However, to prevent any sticking and/or to ensure that the member 30 is completely ejected from the device 1, the projection 24 engages with the section 33 to break any residual engagement stickiness between section 33 and section 16. The used needle member 30 can be safely discarded as a sharp. The device 1 can be reused or, preferably, discarded. Since the device is, in embodiments, essentially a one-piece member, its cost of manufacture is very low. This makes the cost incentive of re-use relatively low. As a result, the risks associated with reusing the device 1 with a new needle member 30 are eliminated and/or reduced. It is submitted that the cheaper the device 1 is to procure, the more likely that the user will use it only once and then discard it.

According to one non-limiting embodiment, the device shown in FIGS. 6 and 7 is packaged. Once the package is open, the user installs the needle member 30 so that the device 1 assumes the configuration of FIG. 1. Again, the is accomplished when the user applies a force F (see FIGS. 10-13) to the member 20 which causes the movable part 25 to move away from the closed position to the open position. At this point, the user can install the member 30 onto the body 10 by inserting the end 35 into the enlarged space formed between parts 15 and 25 and into opening 18, and until the annular surface of the section 32 contacts an annular proximal surface of the parts 15 and 25. The user can then remove the force F and allow the movable part 25 to again assume the position shown in FIGS. 6-9, which results in the device assuming the configuration of FIG. 1. The user can then remove a safety cover (not shown) from the proximal needle 34, inject the same, e.g., into a human or animal, and then, e.g., insert a vacutainer-type vial into the space formed in the body 10. Once the device 1 is utilized to obtain a sample of desired amount and after the sample vial is removed from the interior space of the body 10, and after the injection needle 34 is removed from the injection site, the user can again apply the force F and move the movable part 25 to the open position shown in FIGS. 10-13 and cause ejection of the member 30 as described above.

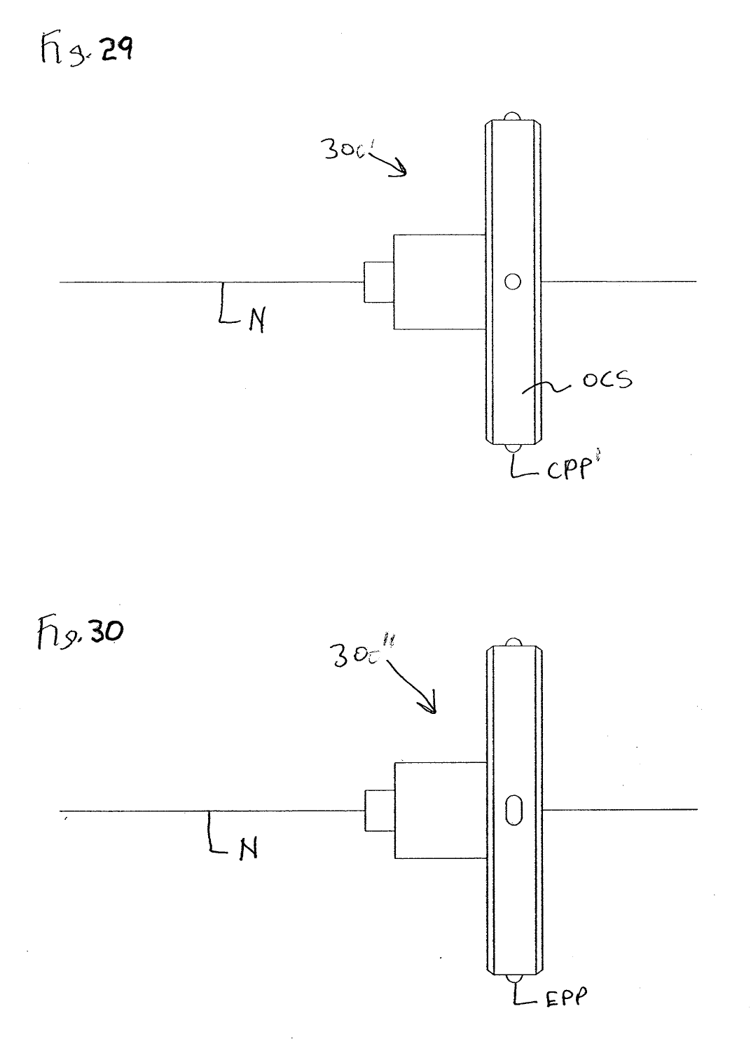

Referring now FIGS. 18-24, there is shown a first non-limiting embodiment of a fluid sampling container 2 which can be utilized with the device 1 of FIG. 1 (see FIG. 19) for injection and/or fluid collection according to the invention. The container 2 functions in a manner similar to a syringe and includes a generally cylindrical sleeve or body member 70. The body 70 has a front generally cylindrical portion 71, a main cylindrical section 72 having an interior space 73 for receiving fluid and a plunger 80, and a flange 74. The front portion 71 is sized and configured to receive thereon and therein a puncturable front cover 90. The plunger 80 has a front piston 81, a main section 82, frangible sections or notches 83, and a flange 84. The container 2 functions in a manner similar to a syringe in that when the user withdraws the plunger 80, fluid is caused to flow into the space 73. However, this will typically occur only when the needle 31 punctures the member 90 (similar to that shown in FIG. 26) and the needle 34 is injected into a source of fluid such as tissue. Before this can happen, however, the user must insert the container 2 into the device 1 as shown in FIG. 19 and fully seat and/or insert the container 2 into the device 1 (similar to that shown in FIG. 26).

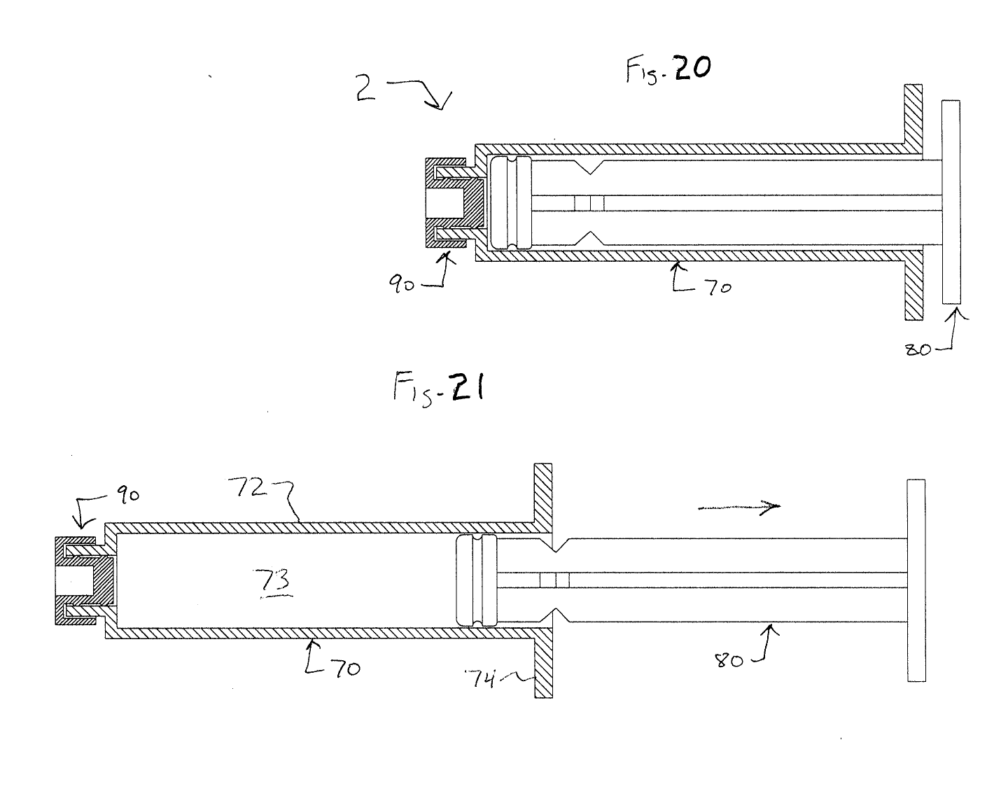

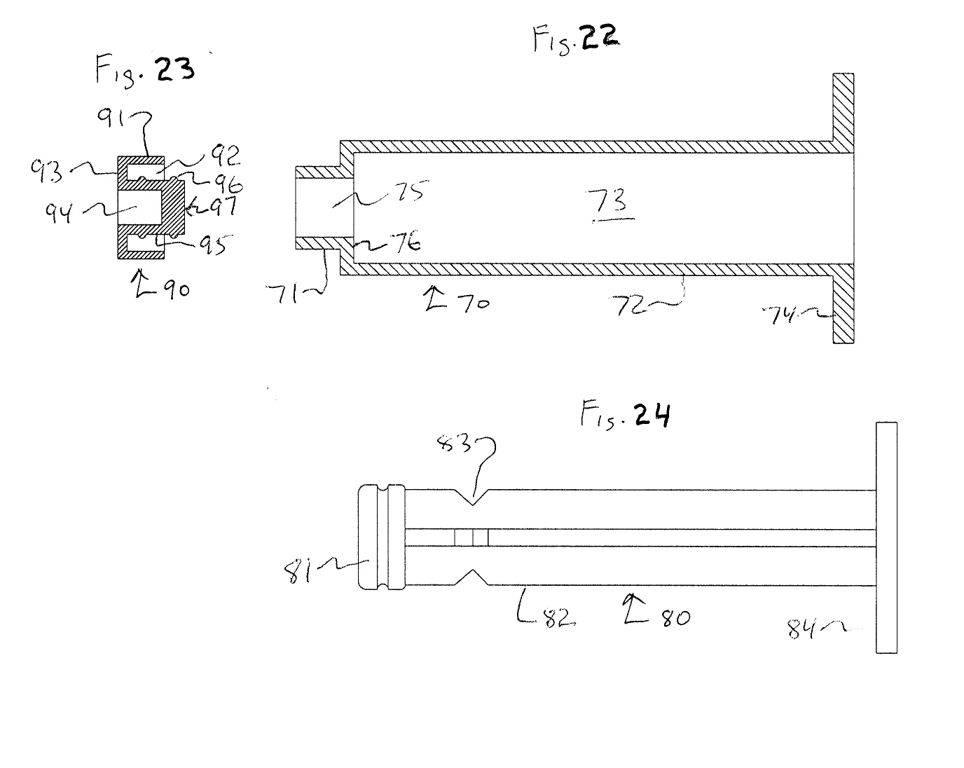

FIGS. 20 and 21 show the fluid sampling container 2 used in the embodiment of FIG. 19. FIG. 20 shows the container 2 in an initial position. In embodiments, this is the position which the container has when packaged and immediately after the packaging is removed. Once the packaging is removed, the user can insert the container 2 into the device 1 as shown in FIG. 19. In this position, the plunger 80 is substantially fully depressed into the body 70 so that little or no dead space is provided between the piston 81 (see FIG. 24) and a spot surface 76 (see FIG. 22). Once the container 2 is fully inserted into the device 1 (which can occur before or after the needle 34 is injected into tissue), the needle 31 will puncture the member 90 and allow for fluid to flow into the space 73. However, this will not occur until the user withdraws the plunger 80 along the direction shown in FIG. 21. An advantage of the invention is that the amount of fluid that flow into the space 73 can be controlled simply by withdrawing the plunger 80 a predetermined amount, i.e., by moving flange 84 away from flange 74. If the user wishes to obtain a small sample, the user can withdraw the plunger 80 relative to the body 70 e.g., only one quarter of the way back. If the user wishes to obtain a larger sample, the user can withdraw the plunger 80 e.g. only half-way back. The largest sample can be obtained by withdrawing the plunger 80 all the way back and/or to it maximum allowed travel backwards. Larger fluid samples can even use multiple containers 2. In the case of the example shown in FIG. 21, the user remove the container 2 from the device 1, and can then break-off the plunger 80 at the frangible section(s) 83 (see FIG. 24) so that the container 2 with the fluid sample contained therein can be transported for analysis and/or stored safely. To remove the fluid from the container 2 for, e.g., analysis, a user can, e.g., simply remove and/or twist-off the member 90 and pour out the fluid.

According to one non-limiting method of using the system of the invention, the user can use the device 1 using conventional vacutainer-type containers. Once sufficient fluid is removed with such containers, one or more an additional samples can be taken using the container 2. This sampling can be used for a different purpose and/or analysis where a precise and/or controlled amount of fluid is desired.

According to another non-limiting method of using the system of the invention, the user can use the device 1 using conventional vacutainer-type containers. Once (or before) sufficient fluid is removed with such containers and/or in between steps in such removal, one or more fluids already arranged in the container 2 (in embodiments, such fluid would flow through a needle passing through the puncturable member 90 or placed/poured into the space 73 via passage 75 before the cover 90 is installed) can be injected into the tissue after the user inserts the container 2 into the device 1. In this case, the container 2 would have the plunger withdrawn configuration shown in, e.g., FIG. 21. In this embodiment, the container 2 (which was previously filled with a desired amount of fluid) is used for injecting a controlled amount of fluid via the device 1 while it is already injected in tissue. Thus, while a patient is being sampled for fluid, e.g., blood, he/she can also be injected with the same or other fluid without requiring the patient to experience any additional injections. The same injection site (via needle 34) is used for both fluid sampling and fluid/medication injection.

FIGS. 22-24 show the main components of the fluid sampling container 2 used in the embodiment of FIG. 19. FIG. 22 shows the body 70 of the fluid sampling container 2. The body 70 has a front cylindrical section 71 which is sized and configured to extend into a space 92 of the puncturable front cover 90. The outer cylindrical surface of the cylindrical section 71 can be in sealing engagement with the inner cylindrical surface of the section 91 of the member 90. The inner cylindrical surface of the cylindrical section 71 is sealing engagement with the outer cylindrical surface 95 of the section 97 of the member 90. Sealing is enhanced and/or ensured via circumferential sealing projections 96. The body 70 also has a stop surface 76 which limits forward movement of the plunger 80 as well as a main fluid space 73 arranged within a generally cylindrical section 72 and a flange 74. In embodiments, the space 73 is such that an amount of fluid substantially equivalent to that contained in a typical vacutainer container is allowed to be taken into the space 73. In embodiments, the space 73 is such that a predetermined amount of fluid substantially equivalent to that contained in a typical vacutainer container (plus a defined percentage or ratio) is allowed to be taken into the space 73. In embodiments, the space 73 is such that a predetermined amount of fluid substantially equivalent to that contained in a typical vacutainer container (minus a defined percentage or ratio) is allowed to be taken into the space 73. The member or front cover 90 has a space 92, an outer cylindrical section 91, an outer cylindrical surface 95 arranged on a puncturable section 97, circumferential sealing projections 96, a front annular section 93 and an injection needle accommodating space 94. The material for the member 90 can be the same as that used for puncturable members on conventional vacutainer-type containers. The plunger 80 has a piston 81 sized and configured to sealingly engage with the inner cylindrical surface of the section 72, a flange 84 arranged on a rear or distal end of the plunger 80, a shaft section 82 which can have the form of cross-shaped ribs or plates as is typically used in single-use syringes, as well as one or more notches 83 which form frangible areas or sections on the shaft 82 and which allows the user snap-off the back portion of the plunger 80.

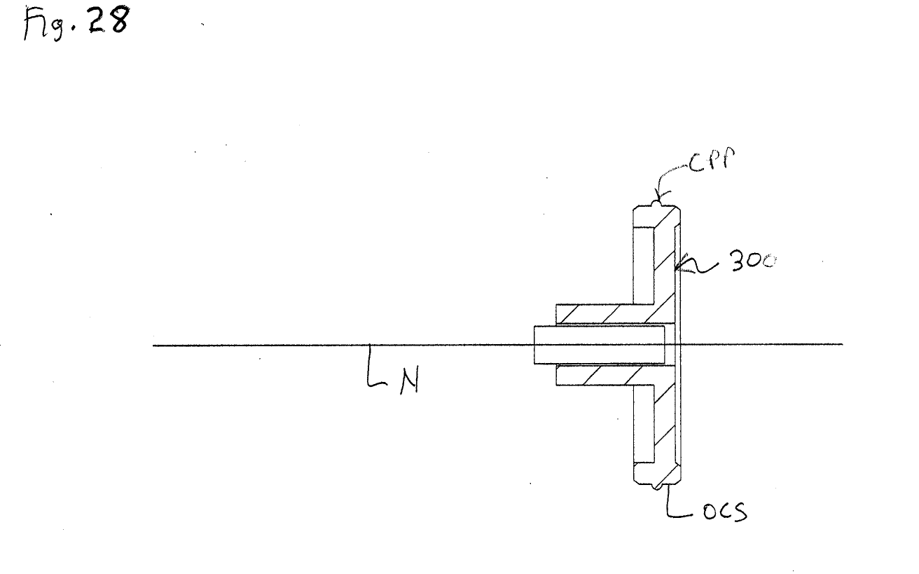

FIG. 25 shows a modified version of a fluid sampling container 2′. The fluid sampling container 2′ is similar to the previous embodiment except that it additionally utilizes a retaining ring RR to help retain the cover 90′ on the section 71′. The body 70′ has a front cylindrical section 71′ which is sized and configured to extend into a space 92′ of the puncturable front cover 90′. The outer cylindrical surface of the cylindrical section 71′ is sealing engagement with the inner cylindrical surface of the section 91′ of the member 90′ and includes a circumferential projection 76′ that engages with a circumferential recess 97′ and functions and an axially retention/sealing system. The inner cylindrical surface of the cylindrical section 71′ is sealing engagement with the outer cylindrical surface 95′ of the section 97′ of the member 90′. Sealing is enhanced and/or ensured via circumferential sealing projections 96′.

FIG. 26 shows a partial side-cross section view another non-limiting embodiment of a fluid sampling system in accordance with the invention. The system utilizes a device 1″ similar to the device of FIG. 1 except that it additionally utilizes a stop projection SP to limit insertion movement of the container 2″ and/or to define the full insertion position thereof. In FIG. 26, the fluid sampling container 2″ is fully inserted into the device 1″ and the rear needle of the needle member 31 has punctured the front cover 90. In this configuration, the user can withdraw the plunger 80 and cause fluid to pass or flow into the container 2″ in the same way as a syringe. The container body 70″ includes a stop surface 76″ to limit forward movement of the plunger 80 and provide sufficient spacing so that the rear needle 31 of the needle member 30 does not contact the piston of the plunger 80.

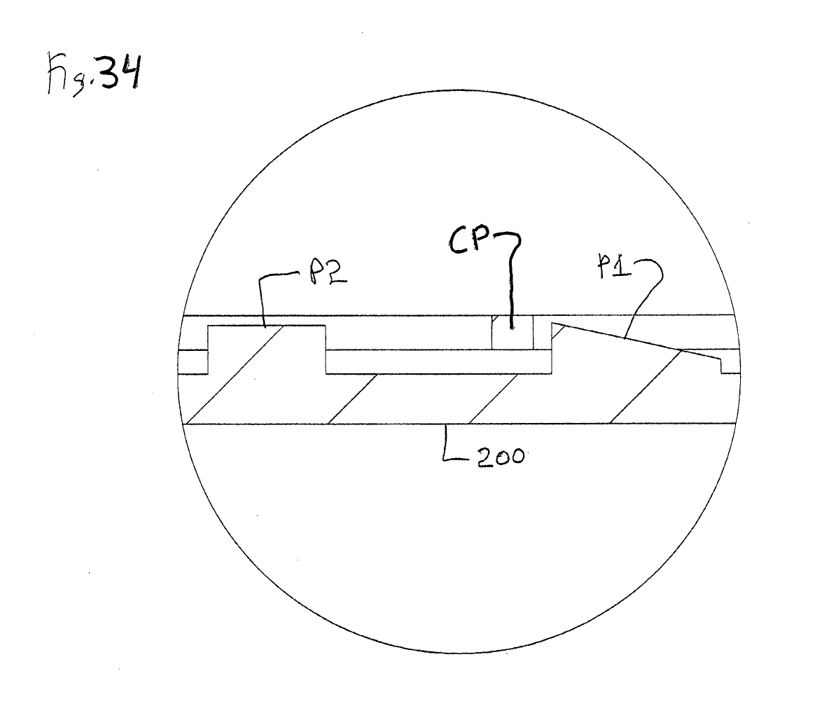

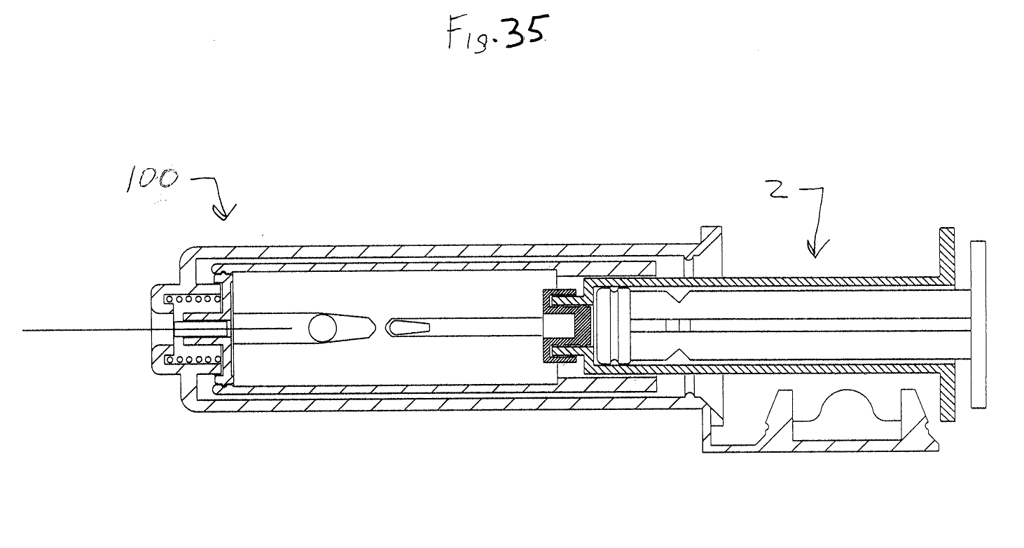

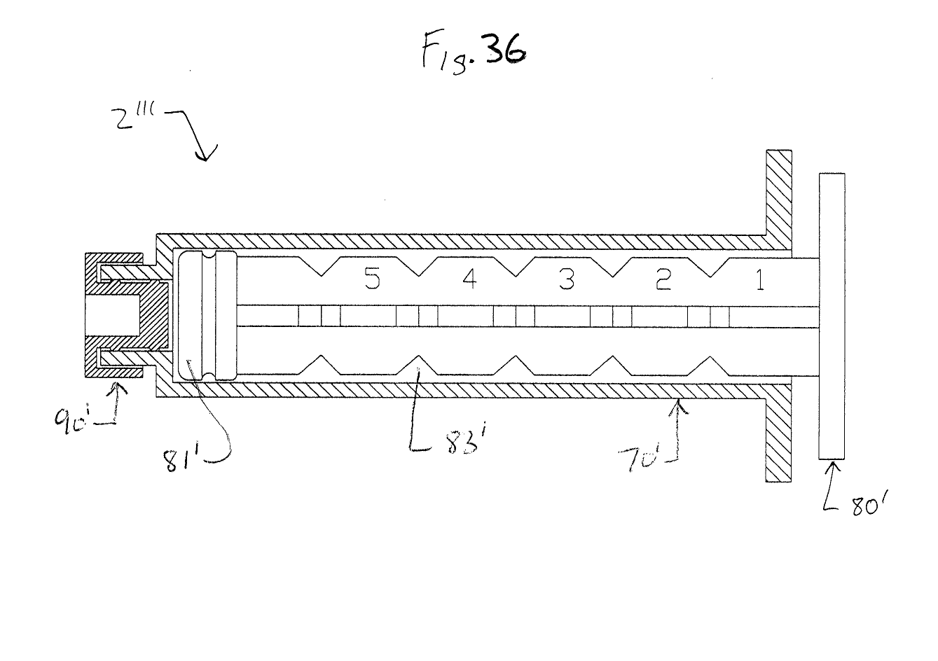

FIGS. 27-35 show another non-limiting embodiment of a blood collection system according to the invention. As with the previous embodiments, the system utilizes a fluid/blood collection device 100 and a syringe-type collection container 2. The device 100 includes a generally cylindrical outer sleeve or outer body member 200 which includes a proximal end 200a configured to allow an external needle N of a double-ended needle member or holder 300 to pass therethrough, and a distal end which can be closed off by a cap 500. An inner flange IF is arranged at the proximal end 200a. The needle holder 300 has an outer circumferential surface OCS which frictionally engages with an inner circumferential surface ICS of a proximal end of an inner sleeve 400. The sleeve 400 has the configuration shown in FIGS. 31-32 and includes proximal slots PS that are made wider than distal slots DS. The surfaces ICS and OCS (see FIGS. 28 and 31) have generally corresponding shapes and need not be straight or cylindrical, i.e., they can also be, e.g., tapered or have an other non-straight shapes. The device 100 also includes a spring 600 which functions to move the needle holder 300 distally when the outer circumferential surface OCS of the needle holder 300 is released from frictional engagement with the inner circumferential surface ICS of the proximal end of the inner sleeve 400. Once a user moves the cap 500 to the closed position, the sleeve 400 is caused to move axially in the proximal direction which, in turn, results in the outer circumferential surface OCS of the needle holder 300 to no longer frictionally engage with the inner circumferential surface ICS of the proximal end of the inner sleeve 400. The spring 600 is then free to move the needle holder 300 within the sleeve 400 in a distal direction which ensures that the needle holder 300 is fully and safely arranged within the device 100. The device can then be safely handled and discarded.

The disengagement of the proximal end of the sleeve 400 from the needle holder 300 functions as follows. The sleeve 400 is prevented from moving axially backwards within the body 200 by the two oppositely arranged projections P1, and is biased towards this distal direction by the spring 600 which, in FIG. 27, is almost fully compressed. The sleeve 400 has a plurality of proximal slots PS (e.g., two) which divide the proximal end of the sleeve 400 into a plurality of spring fingers or portions each having a portion of the surface ICS. The body 200 also includes two oppositely arranged circular projections P2 arranged therein which are each configured to engage (i.e., wedge open) with one of the proximal slots PS of the sleeve 400 when the sleeve 400 is moved in the proximal direction by the closing of the cap 500. Furthermore, when the sleeve 400 is moved in the proximal direction by the closing of the cap 500, the tapered surfaces of the proximal slot PS are contacted the circular surface of the projection P2 and this causes the spring fingers formed by the proximal slots PS to expand radially thereby releasing the engagement between the surface ICS of the sleeve 400 and the surface OCS of the holder 300. As a result, the proximal slots PS widen. This movement of the sleeve 400 and disengagement of the surfaces ICS and OCS is caused to occur automatically when the user moves the cap 500 to the closed position, and more specifically, when an annular surface or proximal end of cap 500 contacts an annular surface or distal end of the sleeve 400 and forces the sleeve 400 to move axially in the proximal direction.

Thus, when the user moves the cap 500 to the closed position (not shown), axial movement of the sleeve 400 automatically causes the surface ICS to substantially radially and circumferentially separate from the surface OCS. Since the frictional engagement between the surface ICS and the surface OCS constitutes the only engagement or connection between the holder 300 and the sleeve 400, the disengagement of these surfaces leaves the holder 300 free to move axially distally. Furthermore, because the spring 600 maintains a biasing force against the holder 300, when this engagement is released, the spring 600 will automatically expand axially and force the holder 300 to move distally within the sleeve 400. This, in turn, results in the needle N being retracted into the sleeve 400 and positions it safely within the body 200. The device 100 is then rendered unusable, i.e., cannot be reused, and can be safely handled and disposed of without fear of the needle causing injury to persons who handle the used device 100.

As was the case with some previous embodiments, the sleeve 400 and the needle holder 300 constitute a sub-assembly. The proximal end of the sleeve 400 forms a plurality of arc-shaped sections or fingers 400a and 400b divided by equally spaced slots PS. The slots PS can be a few as two oppositely arranged slots or as many as, e.g., 20 or more, with any whole number between 2 and 20 being utilized. As such, these fingers are free to deflect outwardly or to slightly elastically deformed outwardly. Furthermore, the spring fingers 400a and 400b can include breakable and/or stretchable connections (similar to connections BEC shown in FIG. 40 of the instant application or FIG. 33 of US 2008/0262421) which are designed to break or stretch when the spring fingers are caused to radially expand beyond a certain point, as occurs when the slots PS engages with the circular projections P2. These integrally formed members BEC also ensure that the fingers are pressed tightly against the holder 300, and more specifically, that the surfaces OSC and ICS remain in engagement until the tapered surfaces of the slots PS engage with the circular projections P2 which allows the fingers to deflect outwardly. In order to assemble this sub-assembly, one need only slide the holder 300 into the proximal end of the sleeve 400. The holder 300 is then prevented from moving axially relative to the sleeve 400 by virtue of engagement between the projection(s) CPP (see FIG. 28) and the corresponding shaped recess(s) or groove(s) in the surface ICS.

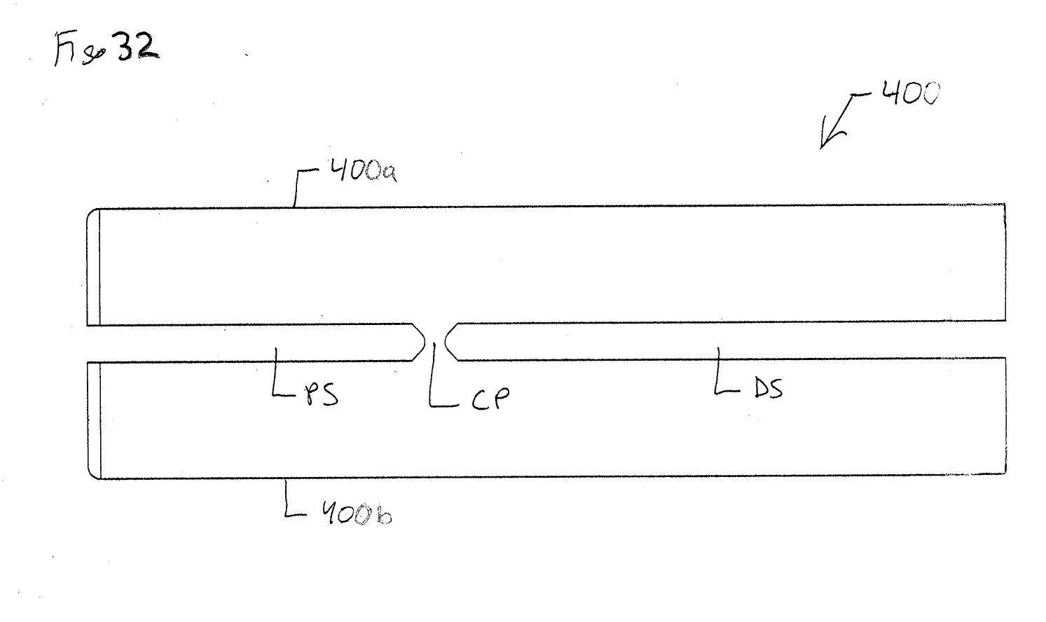

With reference to FIGS. 28-30, it can be seen that the needle holder 300 constitutes a sub-assembly having of holder member having outer surface OCS and a double-ended needle having outer needle N. In the case of FIG. 28, the holder member has a continuous circumferential projection CPP. In the case of FIG. 29, the holder member has four equally angularly spaced circular projections CPP′ which are sized to engage or extend into the groove G of the sleeve 400. Of course, the invention also contemplates using as few as two oppositely arranged projections CPP′. Furthermore, the projections CPP′ need not be circular and/or rounded and can be any shape (e.g., square, triangular, oval, polygonal, etc.) which securely and releasably engages with the groove G. Furthermore, the groove G need not correspond in shape to that of the projections CPP′, and can similarly have any shape which securely and releasably engages with the projections CPP′. By way of non-limiting example FIG. 30 shows four equally angularly spaced elongated projections EPP which are sized to engage or extend into the groove G of the sleeve 400. Again, the invention also contemplates using as few as two oppositely arranged projections EPP. Furthermore, the projections EPP need not be rounded and can be any shape (e.g., square, triangular, oval, polygonal, etc.) which securely and releasably engages with the groove G. The invention also contemplates other mechanisms for providing a releasable engagement between the surfaces OCS and ICS. Still further, instead of merely utilizing a single groove G and a single set of projections CPP′ and EPP, the invention also contemplates using two or more axially spaced grooves G and two or more axially spaced sets of projections CPP′ and EPP.

With reference to FIGS. 31-32, it can be seen that the sleeve 400 has generally half-circular or arc-shaped sections 400a and 400b which together for a sleeve 400 assuming a generally cylindrical shape. The two oppositely arranged connecting portions CP function as the only mechanism connecting together the arc-shaped sections 400a and 400b of the sleeve 400. As such, when the distal ends of these members 400a and 400b are moved towards each other, the connecting portions CP become deflected and/or slightly elastically deformed thereby allowing the proximal ends of the generally half-circular or arc-shaped sections 400a and 400b of the sleeve 400 to move away from each other. However, in the relaxed shown in FIGS. 31 and 32, the connecting portions CP ensure that the surfaces OSC and ICS remain in engagement. In order to assemble the sub-assembly of member 400 and member 300, one need only move the distal ends of the members 400a and 400b towards each other to cause a widening of the slots PS (and simultaneously a narrowing of the slot DS), insert the holder 300 within the proximal end of the sleeve 400, and then remove the force applied to the distal ends allowing the sleeve 400 to again assume a generally cylindrical shape shown in FIGS. 31-32. Of course, proper insertion and connection of the holder 300 requires ensuring that the projection(s) CPP (or CPP′ and EPP) (see FIGS. 28-30) are aligned with the circumferential groove G of the sleeve 400.

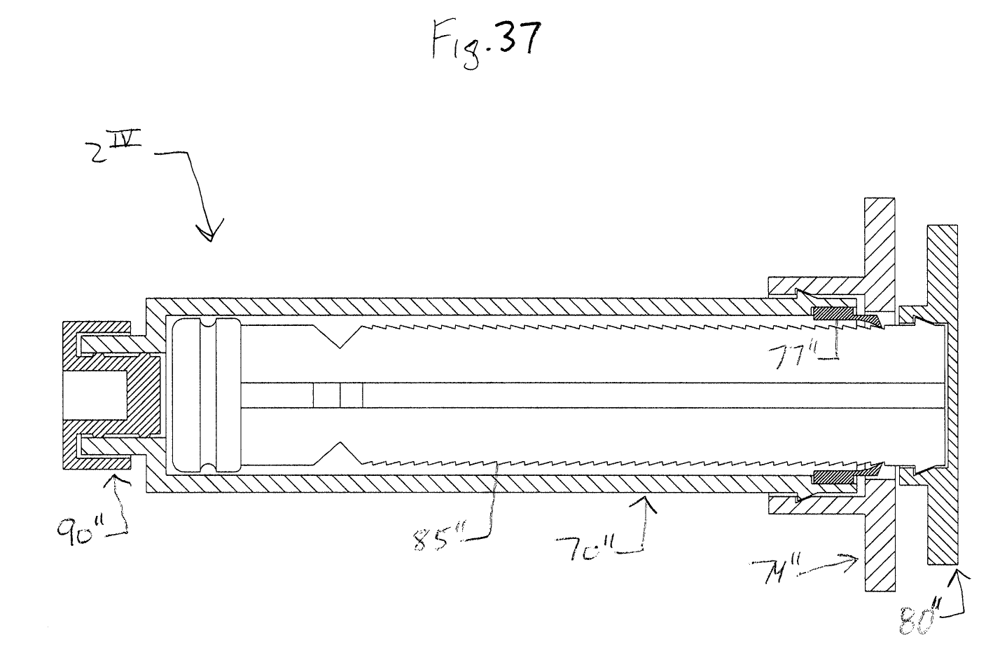

FIG. 35 shows how the user can insert the container 2 into the device 100 described above. After insertion, the user can fully seat and/or insert the container 2 into the device 100 (similar to that shown in FIG. 26 so that the distal needle of member 300 punctured the puncturable member 90). The system shown in FIG. 35 can be used in the same or similar manner described above regarding the embodiment shown in FIG. 19

According to one non-limiting method of using the system of the invention, the user can use the device 100 using conventional vacutainer-type containers. Once sufficient fluid is removed with such containers, one or more an additional samples can be taken using the container 2. This sampling can be used for a different purpose and/or analysis where a precise and/or controlled amount of fluid is desired.

According to another non-limiting method of using the system of the invention, the user can use the device 100 using conventional vacutainer-type containers. Once (or before) sufficient fluid is removed with such containers and/or in between steps in such removal, one or more fluids already arranged in the container 2 (in embodiments, such fluid would flow through a needle passing through the puncturable member 90 or placed/poured into the space 73 via passage 75 before the cover 90 is installed) can be injected into the tissue after the user inserts the container 2 into the device 100. In this case, the container 2 would have the plunger withdrawn configuration shown in, e.g., FIG. 21. In this embodiment, the container 2 (which was previously filled with a desired amount of fluid) is used for injecting a controlled amount of fluid via the device 100 while it is already injected in tissue. Thus, while a patient is being sampled for fluid, e.g., blood, he/she can also be injected with the same or other fluid without requiring the patient to experience any additional injections. The same injection site (via needle N) is used for both fluid sampling and fluid/medication injection. At the end of a procedure, the user can close the cap 500 and render the device 100 single-use and disposable.

FIG. 36 shows a side view of another fluid sample container 2′″ in accordance with another non-limiting embodiment of the invention. The body 70′ and puncturable front cover 90′ are shown in cross-section and are similar to that of the embodiment shown in 18. The plunger 80′ is not shown in cross-section and is shown in an initial and/or fully depresses and/or pre-withdrawn position. In this embodiment, the plunger 80′ has a number of substantially equally axially spaced breakable or frangible sections 83′ as well as indicia so as to provide the user with information as to the quantity of fluid arranged within and/or suctioned into the container 2′″ during withdrawal of the plunger 80′. In operation, the user inserts the container 2′″ into the already injected device 1, 100, etc., and then withdraws the plunger 80′ up to the desired indicia indicator, e.g., 2. Then, he/she deflects the distal end of the plunger 80′ to break it off. The resulting container 2′″ will have stored therein two units of fluid.

FIG. 37 shows a side view of still another fluid sample container 2^{IV} in accordance with still another non-limiting embodiment of the invention. The body 70″, the puncturable front cover 90″, the plunger flange 74″, and the ratchet pawl member 77″ are shown in cross-section. The plunger 80″ is not shown in cross-section and is shown in an initial and/or fully depresses and/or pre-withdrawn position. In this embodiment, the plunger 80″ has a number of ratchet teeth 85″ which are engaged by pawls 77″ so as to prevent the plunger 80″ from being depressed or move proximally after being withdrawn or moved distally. The engagement between teeth 85″ and the pawls 77″ provide a one-way movement mechanism for the plunger 80″ and/or ensures that a patient is not accidentally injected with the fluid that is being the withdrawn. In this embodiment, both the plunger 80″ and the body 70″ have a separately formed flange section 84″ and 74″ that is non-removably axially connected to and/or may or may not rotate freely relative to the plunger 80″ and the body 70″.

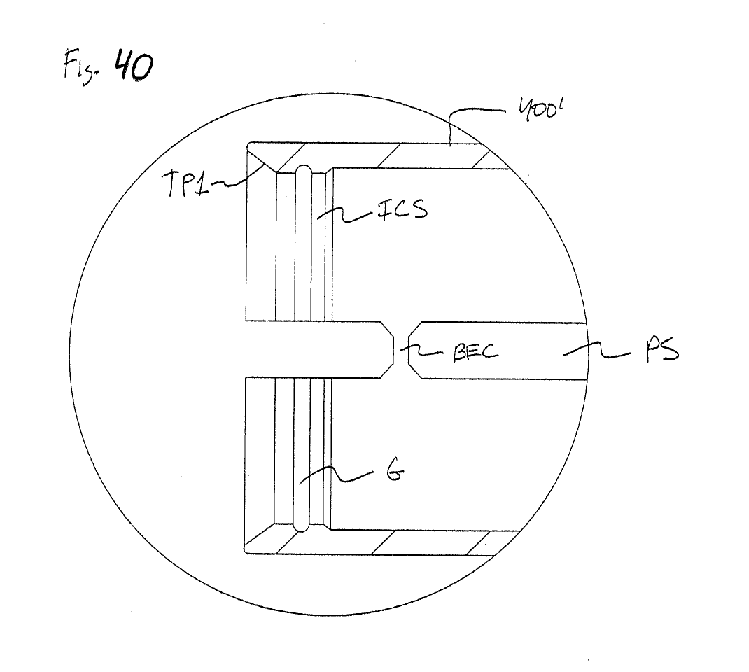

FIGS. 38-40 show another non-limiting embodiment of a blood collection device 100′ according to the invention. The device 100′ includes a generally cylindrical outer sleeve or outer body member 200′ which includes a proximal end 200′ a configured to allow an external needle N of a double-ended needle member or holder 300′ to pass therethrough, and a distal end which can be closed off by a cap 500′. An inner flange IF is arranged at the proximal end 200′a. The needle holder 300′ has an outer circumferential surface OCS which frictionally engages with an inner circumferential surface ICS of a proximal end of an inner sleeve 400′. The surfaces ICS and OCS have generally corresponding shapes and need not be straight or cylindrical, i.e., they can also be, e.g., tapered or have an other non-straight shapes. The device 100′ also includes a spring 600′ which functions to move the needle holder 300′ distally when the outer circumferential surface OCS of the needle holder 300′ is released from frictional engagement with the inner circumferential surface ICS of the proximal end of the inner sleeve 400′. Once a user moves the cap 500′ to the closed position, the sleeve 400′ is caused to move axially in the proximal direction which, in turn, causes outer circumferential surface OCS of the needle holder 300′ no longer frictionally engage with the inner circumferential surface ICS of the proximal end of the inner sleeve 400′. The spring 600′ is then free to move the needle holder 300′ within the sleeve 400′ in a distal direction which ensures that the needle holder 300′ is fully and safely arranged within the device 100′. The device can then be safely handled and discarded.

The disengagement of the proximal end of the sleeve 400′ from the needle holder 300′ functions as follows. The sleeve 400′ is prevented from moving axially backwards within the body 200′ by a circumferential tapered shoulder TS′, but is biased towards this direction by the spring 600′ which, in FIG. 38, is almost fully compressed. The sleeve 400′ has a plurality of proximal slots PS which divide the proximal end of the sleeve 400′ into a plurality of spring fingers each having a portion of the surface ICS. The proximal end of the spring fingers or the sleeve 400′ includes a circumferential tapered portion TP1 which is configured to engage with an annular or circumferential tapered portion TP2 of the body 200′ when the sleeve 400′ is moved in the proximal direction by the cap 500′. Furthermore, when the sleeve 400′ is moved in the proximal direction by the closing of the cap 500′, the tapered surfaces TP1 of the spring fingers of the sleeve 400′ contacts the tapered surface TP2 and causes the spring fingers formed by the proximal slots PS to expand radially thereby releasing the engagement between the surface ICS of the sleeve 400′ and the surface OCS of the holder 300′. As a result, the proximal slots PS widen. This movement of the sleeve 400′ and disengagement of the surfaces ICS and OCS is caused to occur automatically when the user moves the cap 500′ to the closed position, and more specifically, when an annular surface or proximal end of cap 500′ contacts an annular surface or distal end of the sleeve 400′ and forces the sleeve 400′ to move axially in the proximal direction.

Thus, when the user moves the cap 500′ to the closed position (not shown), axial movement of the sleeve 400′ automatically causes the surface ICS to separate from the surface OCS. Since the frictional engagement between the surface ICS and the surface OCS constitutes the only engagement or connection between the holder 300′ and the sleeve 400′, the disengagement of these surfaces leaves the holder 300′ free to move axially distally. Furthermore, because the spring 600′ maintains a biasing force against the holder 300′, when this engagement is released, the spring 600′ will automatically expand axially and force the holder 300′ to move distally within the sleeve 400′. This, in turn, results in the needle N being retracted into the sleeve 400′ and positions it safely within the body 200′. The device 100′ is then rendered unusable, i.e., cannot be reused, and can be safely handled and disposed of without fear of the needle N causing injury to persons who handle the used device 100′.

As was the case with some previous embodiments, the sleeve 400′ and the needle holder 300′ constitute a sub-assembly. The proximal end of the sleeve 400′ forms a plurality of arc-shaped sections or fingers 400′a and 400′b divided by equally spaced slots PS. The slots PS can be a few as two oppositely arranged slots or as many as, e.g., 20 or more, with any whole number between 2 and 20 being utilized. As such, these fingers are free to deflect outwardly or to slightly elastically deformed outwardly. However, the spring fingers of the member 400′ are also preferably connected to each other via breakable and/or stretchable connections BEC (see FIG. 40) which are designed to break or stretch when the spring fingers are caused to radially expand beyond a certain point, as occurs when the surface TP1 engages with the surface TP2. These integrally formed members BEC also ensure that the fingers are pressed tightly against the holder 300′, and more specifically, that the surfaces OSC and ICS remain in engagement until the surface TP1 engages significantly with the surface TP2 which allows the fingers to deflect outwardly. In order to assemble this sub-assembly, one need only slide the holder 300′ into the proximal end of the sleeve 400′. The holder 300′ is then prevented from moving axially relative to the sleeve 400′ by virtue of engagement between the projections CPP (see FIG. 28) and the corresponding shaped recess(s) or groove(s) in the surface ICS.

The devices described herein can also utilize one or more features disclosed in the prior art documents expressly incorporated by reference herein. Furthermore, one or more of the various parts of the device can preferably be made as one-piece structures by e.g., injection molding, when doing so reduces costs of manufacture. Non-limiting materials for most of the parts include synthetic resins such as those approved for syringes, blood collection devices, or other medical devices. Furthermore, the invention also contemplates that any or all disclosed features of one embodiment may be used on other disclosed embodiments, to the extent such modifications function for their intended purpose.

The devices described herein can also utilize one or more features disclosed in the prior art documents expressly incorporated by reference herein. Furthermore, one or more of the various parts of the device can preferably be made as one-piece structures by e.g., injection molding, when doing so reduces costs of manufacture. Non-limiting materials for most of the parts include synthetic resins such as those approved for syringes, blood collection devices, or other medical devices. Furthermore, the invention also contemplates that any or all disclosed features of one embodiment may be used on other disclosed embodiments, to the extent such modifications function for their intended purpose.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. A fluid collection/injection device comprising:
a body having a front end, a back end, and a main hollow section arranged between the front and back ends;
a needle hub securing section arranged on the front end and being structured and arranged to receive therein a needle member;
a double-ended needle removably and/or movably coupled to the needle hub securing section and/or removably coupled to a retractable needle hub; and
a syringe sampling device having an insertable end which is puncturable by one needle of the double-ended needle.

2. The device of claim 1, wherein the syringe sampling device comprises a body and a plunger having a forward end in sealing engagement with an inner surface of the body.

3. The device of claim 2, wherein the plunger has a rear end configured to allow a user to move the plunger in a rear-ward direction so as to limit an amount of fluid that is withdrawn with the device.

4. The device of claim 2, wherein the plunger has a piston and a breakable and/or frangible and/or separable section.

5. The device of claim 1, wherein the insertable end of the syringe sampling device comprises a re-sealable member that prevents fluid from leaking out of the syringe sampling device when the one needle is withdrawn.

6. The device of claim 1, wherein the syringe sampling device comprises a plunger having a piston and a plurality of breakable and/or frangible sections.

7. The device of claim 1, wherein the syringe sampling device comprises a mechanism that prevents forward movement of a plunger and allows rearward movement of the plunger.

8. The device of claim 1, wherein the syringe sampling device comprises a ratchet mechanism that prevents forward movement of a plunger and allows rearward movement of the plunger.

9. The device of claim 1, wherein the insertable end of the syringe sampling device comprises a removable re-sealable member that prevents fluid from leaking out of the syringe sampling device when the one needle is withdrawn.

10. The device of claim 1, wherein the body, the needle hub securing section, and the double ended needle is a conventional device.

11. The device of claim 1, wherein the body and the needle hub securing section comprise a one-piece member.

12. The device of claim 1, wherein the body and the needle hub securing section comprise separate members.

13. A method of taking a fluid sample using the device of claim 1, the method comprising:
inserting the syringe sampling device into the body; and
removing the syringe sampling device from the body.

14. A method of taking a fluid sample using the device of claim 1, the method comprising:
inserting the syringe sampling device into the body;
withdrawing a plunger of the syringe sampling device by a desired amount; and
removing the syringe sampling device from the body.

15. A fluid collection/injection system comprising:
a fluid sampling syringe having a double-ended needle arranged on a forward end and a rear end;
a syringe sampling device having a forward end insertable into the rear end; and
the forward end of the syringe sampling device being puncturable by one needle of the double-ended needle.

16. A method of taking a fluid sample using the device or system of claim 15, the method comprising:
inserting the syringe sampling device; and
removing the syringe sampling device.

17. A method of taking a fluid sample using the device or system of claim 15, the method comprising:
inserting the syringe sampling device;
withdrawing a plunger of the syringe sampling device by a desired amount; and
removing the syringe sampling device.

18. A fluid collection/injection system comprising:
a fluid sampling body having a forward end;
at least one of:
a retractable double-ended needle; and
a removable double-ended needle;
a sampling device insertable into the fluid sampling body; and
the sampling device comprising a movable plunger and a puncturable forward end that is puncturable by one needle of the double-ended needle.

19. The system of claim 18, wherein the sampling device comprises a body and the plunger comprises a forward end in sealing engagement with an inner surface of the body.

20. The system of claim 19, wherein the plunger has a rear end configured to allow a user to move the plunger in a rear-ward direction so as to limit an amount of fluid that is withdrawn with the sampling device.
